(19) United States
(12) Patent Application Publication
(10) Pub. No.: US 2025/0179508 A1
(43) Pub. Date: Jun. 5, 2025

(54) COMPOSITIONS AND METHODS FOR DELIVERY OF NUCLEIC ACIDS BY VIRUS PARTICLES IN PLANTS

(71) Applicant: The Regents of the University of California, Oakland, CA (US)

(72) Inventors: Juan Pablo Giraldo Gomez, Redlands, CA (US); Md Reyazul Islam, Riverside, CA (US); Hye-In Kim, Riverside, CA (US); Nicole F. Steinmetz, San Diego, CA (US); Marina Youngblood, Riverside, CA (US)

(21) Appl. No.: 18/961,937

(22) Filed: Nov. 27, 2024

Related U.S. Application Data
(60) Provisional application No. 63/604,402, filed on Nov. 30, 2023.

(51) Int. Cl. C12N 15/82; B82Y 5/00

(52) U.S. Cl. CPC C12N 15/8203; B82Y 5/00

(57) ABSTRACT

Certain embodiments of the invention provide compositions and methods for nucleic acid delivery by engineered virus-like nanocarriers in plant cells. Certain embodiments of the invention provide a nanoparticle, comprising a cationic polymer (e.g., poly-(allylamine) or salt thereof) conjugated to the nanoparticle, wherein the nanoparticle is a live virus (e.g., TMGMV), or an inactivated virus (e.g., iTMGMV).

### CROSS-REFERENCE TO RELATED APPLICATION

This application claims priority to U.S. Provisional Application No. 63/604,402 that was filed on Nov. 30, 2023. The entire content of the application referenced above is hereby incorporated by reference herein.

### GOVERNMENT FUNDING

This invention was made with government support under 2134535 awarded by the National Science Foundation. The government has certain rights in the invention.

### BACKGROUND

Despite numerous biotechnological advancements over the past decades, the genetic transformation of many plant species still poses considerable challenges. The delivery of transgenes into plant species mainly relies on two transformation methods: Agrobacterium tumefaciens-mediated transformation system and particle bombardment. Current methods used for DNA delivery to plants have limitations and significant drawbacks. New and efficient delivery compositions and methods are needed.

### SUMMARY OF THE INVENTION

Certain embodiments of the invention provide a nanoparticle as described herein, for example, a nanoparticle comprising a cationic polymer (e.g., poly-(allylamine) or salt thereof) conjugated to the nanoparticle, wherein the nanoparticle is a live virus, or an inactivated virus. In certain embodiments, the nanoparticle is selected from the group consisting of tobacco mosaic virus, tobacco mild green mosaic virus, cowpea mosaic virus, and potato virus X. In certain embodiments, a nucleic acid (e.g., DNA or RNA) is adsorbed on the nanoparticle.

Certain embodiments of the invention provide a method for delivering nucleic acid to a plant cell, comprising contacting the plant cell with a nanoparticle as described herein (e.g., contacted in vitro, or in vivo).

Certain embodiments of the invention provide a composition as described herein (e.g., a composition comprising one or more nanoparticle(s) described herein, such as a live virus, or an inactivated virus, or combination thereof).

Certain embodiments of the invention provide a method (e.g., method of making or using) as described herein.

Certain embodiments of the invention provide a conjugate described herein, e.g., a protein-polymer conjugate comprising a viral protein (such as a viral coat protein) and a cationic polymer (e.g., PAH) conjugated to the viral protein. In certain embodiments, the viral protein is derived from tobacco mosaic virus, tobacco mild green mosaic virus, cowpea mosaic virus, potato virus X, or a combination thereof.

Certain embodiments of the invention provide a nanoparticle, comprising a protein-polymer conjugate described herein. In certain embodiments, nucleic acid (e.g., DNA or RNA) is adsorbed on the nanoparticle outer surface.

Certain embodiments of the invention provide a conjugate described herein, e.g., a protein-polymer conjugate comprising Tobacco mild green mosaic virus (TMGMV) coat protein (CP) and poly(allylamine) or salt thereof linked via an amide bond.

Certain embodiments of the invention provide a nanoparticle-polymer conjugate, comprising a nanoparticle, and a cationic polymer (e.g., PAH) conjugated to the nanoparticle, wherein the nanoparticle is a live virus, or an inactivated virus. In certain embodiments, the nanoparticle is tobacco mosaic virus, tobacco mild green mosaic virus, cowpea mosaic virus, potato virus X, or a combination thereof. In certain embodiments, nucleic acid (e.g., DNA or RNA) is adsorbed on the nanoparticle cationic outer surface through electrostatic interactions. In certain embodiments, the nanoparticle is a live virus. In certain embodiments, the nanoparticle is an inactivated virus.

Certain embodiments of the invention provide a TMGMV nanoparticle-polymer conjugate, comprising TMGMV nanoparticle and poly(allylamine) or salt thereof.

### BRIEF DESCRIPTION OF THE FIGURES

FIG. 1. Intracellular DNA delivery in Arabidopsis plant protoplasts mediated by virus-like nanocarriers. Negatively charged TMGMV were coated with a biopolymer, poly-(allylamine) hydrochloride (PAH), imparting them with positive charge (PAH-TMGMV). The PAH-TMGMV were loaded by electrostatics with a DNA oligo (GT₁₅, 30 bp ssDNA) that was covalently linked to a Cy3 organic dye (PAH-TMGMV-GT₁₅-Cy3), or a plasmid DNA (pDNA) encoding a reporter gene of a green fluorescent protein. The nanocarriers and DNA cargoes spontaneously enter plant cell membranes without mechanical aid through energy independent uptake mechanisms. Created using BioRender.com.

FIGS. 2A-2D. Characterization of virus-like nanocarriers coated with single stranded DNA. (FIG. 2A), Transmission electron microscopy of negative-stained TMGMV and PAH-TMGMV. Scale bars 100 nm. Yellow arrows indicate PAH coated on the surface of TMGMV. (FIG. 2B) Denaturing Nu-PAGE gel electrophoresis under white light followed by Coomassie staining, 1: TMGMV, 2: PAH-TMGMV, 3: PAH-TMGMV-GT₁₅-Cy3, M: pre-stained molecular weight standards. The arrow indicates the position of the TMGMV coat protein (CP) at 17.5 kDa (Lower arrow) and PAH conjugated PAH-TMGMV CP at 35 kDa (Upper arrow) or higher molecular weight. (FIG. 2C) UV-Vis absorbance and (FIG. 2D) zeta potential (10 mM MES, pH 6.0) of TMGMV before and after coating with PAH and GT₁₅-Cy3. The data are the means±SD (n=4). Statistical analysis was performed by one-way analysis of variance (ANOVA) with Tukey's post-hoc multiple comparison analysis (GraphPad Prism 6); *P<0.015; ****P<0.0001

FIGS. 3A-3D. Delivery of single stranded DNA by virus-like nanocarriers in plant protoplasts. (FIG. 3A) Confocal images of isolated mesophyll protoplasts with chlorophyll autofluorescence (magenta) exposed to PAH-TMGMV-GT₁₅-Cy3 (0.1 mg/mL). The GT₁₅-Cy3 was detected in protoplast membranes (white arrows) and nuclei (yellow arrows). Scale bars 30 μm. (FIG. 3B) After treatment with PAH-TMGMV-GT₁₅-Cy3, protoplasts were stained either with a nuclear marker, Hoechst, or cell membrane staining dye, FM-4-64 for confocal microscopy imaging. Scale bars 5 μm. (FIG. 3C) Orthogonal projections from z-stacks of different planes (x/y, x/z, or y/z) of confocal microscopy images indicating localization of GT₁₅-Cy3 with Hoechst nuclear marker. Scale bars 30 μm. (FIG. 3D) Quantitative analysis of subcellular localization of GT₁₅-Cy3 with Hoechst nuclear marker and FM-4-64 cell membrane dye. The data are means±SD (n=3).

FIGS. 4A-4E. Plasmid DNA delivery and expression mediated by virus-like nanocarriers in isolated plant protoplasts. (FIG. 4A) DNA loading analysis by agarose gel electrophoresis of pDNA (p35S-eGFP) bound to PAH-TMGMV at mass ratios 1:1 to 1:30. M:DNA ladder. Black arrows indicate supercoiled (below) and circular (upper) pDNA bands. The red arrow indicates pDNA bound to PAH-TMGMV that prevents its mobility through the gel. (FIG. 4B) Zeta potential measurements of virus-like nanocarriers with or without pDNA (10 mM MES, pH 6.0). Data are means±SD (n=3-4). (FIG. 4C) Representative TEM images of TMGMV, PAH-TMGMV, and pDNA-loaded at 1:6 mass ratios to PAH-TMGMV. Scale bar 100 nm. Arrows indicate pDNA attachment to PAH-TMGMV. (FIG. 4D) pDNA delivery and expression mediated by PAH-TMGMV in isolated plant protoplasts determined by confocal microscopy. Scale bar 10 μm. (FIG. 4E) GFP expression analysis by western blotting. The arrow indicates 27 kDa of GFP protein and asterisks indicate non-specific bands. M, protein ladder.

FIGS. 5A-5D. Protoplast cell viability after exposure to DNA-coated virus-like nanocarriers. Isolated Arabidopsis protoplasts were treated with different concentrations of TMGMV-PAH coated in GT15-Cy3 or pDNA for 4 hours and subsequently stained with 0.01% fluorescein diacetate (FDA). Confocal and brightfield microscopy images of FDA-treated protoplast cells incubated with (FIG. 5A) different concentrations of TMGMV-PAH (0.1-0.5 mg/mL) coated in GT15-Cy3, (FIG. 5B) TMGMV-PAH loaded with various mass ratios of pDNA (TMGMV-PAH:pDNA=1:3, 1:6, 1:12). Scale bars 25 m. Quantitative analysis of protoplast cell viability after exposure to (FIG. 5C) TMGMV-PAH-GT15-Cy3 and (FIG. 5D) TMGMV-PAH loaded with pDNA (TMGMV-PAH:pDNA) at various mass ratios 1:3, 1:6, and 1:12. The data are the means±SD (n=3). The statistical analysis performed compares with protoplasts-only signals using a one-way ANOVA and post-hoc Tukey test, *p<0.02, ***p<0.0003, ****p<0.0001, and ns: nonsignificant.

FIG. 6. Surface functionalization of TMGMVs using poly (allylamine) hydrochloride (PAH). TMGMV were coated with PAH to create positively charged nanocarriers (PAH-TMGMV) for DNA delivery. After the conjugation reaction, nanocarriers were purified using a 100 kDa MWCO (molecular weight cutoff) dialysis membrane to remove unreacted EDC/NHS, PAH, and broken nanomaterials. Subsequently, aggregated particles were removed using centrifugation, and the collected supernatant of monodispersed PAH-TMGMV.

FIG. 7. FTIR analysis of PAH, TMGMV, and PAH-TMGMV. The FTIR spectra analysis was conducted to determine the covalent modification of TMGMVs with the PAH polymer through carbodiimide chemistry. The primary amine of PAH, involved in the carbodiimide conjugation, showed a noticeable decrease in transmittance, resulting in an enhanced secondary amide peak in PAH-TMGMVs. Characteristic peaks at 1601 cm⁻¹ and 1506 cm⁻¹, representing the symmetric and asymmetric N—H deformation of the protonated primary amine on the PAH polymer were reduced after conjugation. The resultant secondary amide, formed between PAH and TMGMV, displayed its trans- and cis-form C—N stretching at 1251-1205 cm⁻¹ and 1358-1308 cm⁻¹, respectively, with its N—H bending vibration for the secondary amine at 1452 cm⁻¹. Typical secondary amides showcased strong bands around 3270 cm⁻¹ and within the 3100-3070 cm⁻¹ range. The cis- and trans-configurations of these secondary amides can be discerned from their respective hydrogen-bonded N—H vibration bands at 3417 and 3290 cm⁻¹, respectively. Collectively, the FTIR spectra transition from the primary amine in PAH to secondary amide features in PAH-TMGMVs underscore the covalent modification of TMGMV by PAH.

FIGS. 8A-8C. Characterization of PAH-TMGMV and PAH-TMGMV-GT₁₅-Cy3. (FIG. 8A) After conjugation of GT₁₅-Cy3 to the PAH-TMGMV, the sample was purified using a centrifugal filter unit (100 K MWCO) and washed 4 times using 10 mM MES, pH-6.0 following centrifugation at 12000×g, 10 min. Eluent and washout samples were measured with a UV-Vis spectrophotometer to confirm the absence of free dye. No absorbance of GT₁₅-Cy3 was observed after the second wash. (FIG. 8B) Fluorescence spectra of PAH-TMGMV, GT₁₅-Cy3, and PAH-TMGMV-GT₁₅-Cy3. (FIG. 8C) The hydrodynamic diameter of TMGMV, PAH-TMGMV, and PAH-TMGMV-GT₁₅-Cy3 was measured via dynamic light scattering (DLS) in 10 mM MES buffer, pH-6.0. The data represent the mean, (n=4). Statistical analysis was performed by one-way analysis of variance (ANOVA) with Tukey's post-hoc multiple comparison analysis (GraphPad Prism 6); P<0.05.

FIG. 9. Isolation of mesophyll protoplasts from Arabidopsis thaliana leaves. Protoplasts were isolated from 2-3 weeks-old Arabidopsis seedlings using an enzymatic digestion method of leaf tissues. The protoplasts were purified at room temperature by a combination of filtration, 21% sucrose gradient centrifugation, and washing. Chlorophyll autofluorescence (left), brightfield image (middle), and merge image (right) were collected by confocal microscopy. Scale bar 30 μm.

FIG. 10. DNA delivery (GT15-Cy3) by PAH-TMGMV in Arabidopsis mesophyll protoplasts. Isolated mesophyll protoplast cells were treated with 0.1 mg/mL of PAH-TMGMV-GT₁₅-Cy3 and imaged with confocal microscopy. (a) Nuclear localization was confirmed by staining with a nuclear marker, Hoechst, and merged with GT₁₅-Cy3 channels. Scale bars 30 μm.

FIGS. 11A-11B. Delivery mechanism of DNA mediated by PAH-TMGMV in isolated plant protoplast. (FIG. 11A) Cellular uptake of PAH-TMGMV-GT₁₅-Cy3 at 25° C. and 4° C. A volume of 100 μL of isolated protoplast in W5 solution, containing 1.9×10⁵ cells mL-1, was separately treated with 0.1 mg/mL of PAH-TMGMV-GT₁₅-Cy3 at 25° C. and 4° C. for 1 hour. For the 4° C. incubation, protoplasts were pre-treated at 4° C. for 20 min before being exposed to PAH-TMGMV-GT₁₅-Cy3, and an ice-cold W5 buffer was used. Following incubation, protoplasts were washed at 25° C. and 4° C. with W5 buffer using a centrifuge of 100×g for 2 min. The supernatant was removed, and the protoplasts were resuspended in 100 μL of W5 buffer. For confocal microscopy imaging, 20 μl of cell suspension was placed with a poly-lysin-coated glass slide, incubated for 20 mins to allow for cell precipitation at 25° C. and 4° C., the buffer was carefully removed, and 20 μl of ice-cold or room-temperature 50% glycerol diluted with W5 buffer was added. Scale bars 30 μm. All images share the same scale bar. (FIG. 11B) Quantitative analysis of cellular uptake at 25° C. and 4° C. Cellular uptake percentages of PAH-TMGMV-GT₁₅-Cy3 at 25° C. and 4° C. were calculated based on the confocal images of Cy3 fluorescence signal found only inside the intact protoplast cells (indicated by yellow arrows) out of the total number of intact protoplasts determined from the bright field images. The data are the means±SD (n=3). The statistical analysis was performed using a one-way ANOVA with Tukey's post-hoc multiple comparison analysis (GraphPad Prism 6), ns: non-significant.

FIGS. 12A-12B. The p35S-eGFP plasmid encodes a green fluorescence protein with a CaMV 35S promoter. (FIG. 12A) SnapGene software was used to generate a plasmid map from the p35S-eGFP nucleotide sequence. (FIG. 12B) Gel electrophoresis analysis of isolated p35S-eGFP. The p35S-eGFP plasmid was isolated and purified from transformed E. coli DH5α cells, and concentration and purity were measured by Nanodrop (2000c) spectrophotometers. A 300 ng of p35S-eGFP was run in 1% agarose gel electrophoresis. The arrow indicates the p35S-eGFP plasmid band (4.1 kb). M, 1 Kb plus DNA ladder.

FIG. 13. TEM images of negative-stained PAH-TMGMV with pDNA. The PAH-TMGMV were loaded at various mass ratios; PAH-TMGMV/pDNA=1:1, 1:3, 1:12 to PAH-TMGMV nanocarriers. Arrows indicate pDNA attachment to PAH-TMGMV. Scale bars, 100 nm.

FIG. 14. Protection of pDNA (p35S-eGFP) from DNase activity by PAH-TMGMV nanocarriers indicated by electrophoresis analysis. The pDNA band analysis on a 1% agarose gel electrophoresis of pDNA (600 g), PAH-TMGMV (100 ng), or pDNA loaded onto PAH-TMGMV at PAH-TMGMV:pDNA mass ratio of 1:6 (w w), with DNase I (+) and without DNase I (−) (30 min at 37° C.). The black arrow indicates supercoiled (below) and circular (upper) plasmid p35S-eGFP bands. The red arrow indicates pDNA bound to PAH-TMGMV nanocarriers, preventing its movement in the agarose gel. Both DNase I treated (+) and untreated (−) PAH-TMGMV-pDNA samples showed the same band intensity for pDNA (red arrow), suggesting that pDNA is well protected from DNase I. In contrast, pDNA alone treated with DNase I showed no detectable DNA bands, indicating that the pDNA alone was completely digested by DNase I. M represents the DNA ladder.

FIGS. 15A-15B. Protoplast cell viability in the presence of DNA-coated virus-like nanocarriers. Confocal and brightfield microscopy images of FDA-treated protoplast cells incubated with different concentrations of PAH-TMGMV (0.2-0.5 mg/mL) coated in GT₁₅-Cy3 (FIG. 15A), and PAH-TMGMV loaded with various mass ratios of pDNA (PAH-TMGMV:pDNA=1:3, 1:12) (FIG. 15B). Incubation time was 4 hours followed by staining with 0.01% fluorescein diacetate (FDA). Scale bars 25 μm.

FIGS. 16A-16C. Plasmid DNA delivery and expression mediated by iTMGMV-PAH-pDNA in Arabidopsis leaves. Green fluorescence protein (GFP) (FIG. 16A) confocal microscopy images and (FIG. 16B) and fluorescence intensity (n=3) indicating GFP expression in leaf epidermal cells infiltrated with iTMGMVPAH-pDNA. Three-week-old Arabidopsis leaves were abaxially infiltrated with (1:6) 0.1 mg/mL iTMGMV-PAH: 0.6 mg/mL pDNA and analyzed 2 days post infiltration (n=3). Scale bars 30 μm. One-way ANOVA with Tukey's posthoc multiple comparison analysis; ****P<0.0001. (FIG. 16C) RTqPCR analysis of GFP mRNA expression levels 2 days post iTMGMV-PAH-pDNA infiltration in Arabidopsis leaves. Statistical analysis was performed by one-way ANOVA with Tukey's posthoc multiple comparison analysis; **P<0.005 (n=3).

FIGS. 17A-17B. Comparison of inactivated iTMGMV and active TMGMV. (FIG. 17A) Transmission electron microscopy of negative-stained active TMGMV and inactive iTMGMV, and 0.1 mg/mL iTMGMV-PAH: 0.6 mg/mL pDNA. (FIG. 17B) Zeta potential of active TMGMV and iTMGMV before and after coating with PAH and pDNA. The data are the means±SD (n=6). Statistical analysis was performed by one-way analysis of variance (ANOVA) with Tukey's post-hoc multiple comparison analysis, ****P<0.0001.

FIG. 18. GFP analysis of leaves infiltrated with buffer and iTMGMV-PAH controls. No GFP expression was observed in Arabidopsis epidermal cells infiltrated with buffer or iTMGMV-PAH using confocal microscopy analysis. 3 week-old Arabidopsis leaves were abaxially infiltrated with buffer and iTMGMV-PAH and imaged 2 days post infiltration (n=3). Scale bars 30 m.

FIGS. 19A-19B. Leaf cell viability after exposure to DNA-coated virus-like nanocarriers. Arabidopsis leaf discs were treated with 0.1 mM propidium iodide for 30 minutes. Confocal and brightfield images of (FIG. 19A) buffer, pDNA, iTMGMV-PAH and three different concentrations of 1:6 iTMGMV-PAH coated with pDNA. White arrows show stained nuclei indicating a dead cell. Scale bars 100 m. (FIG. 19B) Quantitative analysis of leaf cell viability after exposure to three different concentrations of 1:6 iTMGMV-PAH-pDNA. The data are the means±SD (n=3). The statistical analysis performed a one-way ANOVA with Tukey's post-hoc multiple comparison analysis, *p=0.017.

FIG. 20. Zeta potential of cationic polymer-coated TMGMVs. TMGMVs were functionalized with various cationic polymers: poly-L-lysine (TMGMV-PLL), polyarginine (TMGMV-pArg), and poly-allylamine hydrochloride (TMGMV-PAH). The zeta potential of these functionalized TMGMVs (10 g/mL) were analyzed using a zetasizer at pH 6.0 in the presence of 0.1 mM NaCl in a 10 mM MES buffer. Coating TMGMVs with PAH polymers results in highly charged TMGMV-PAH particles.

### DETAILED DESCRIPTION

Certain embodiments of the invention provide engineered virus-like nanocarriers for efficient delivery of one or more nucleic acids into plant cell(s). Accordingly, one aspect of the invention provides a nanoparticle comprising a cationic polymer conjugated to the nanoparticle, wherein the nanoparticle is a live virus, or an inactivated virus. The engineered virus like nanoparticle may have a cationic surface suitable for adsorption of nucleic acid(s) through electrostatic interactions. Such engineered nanocarriers as shown herein have one of the highest nucleic acid mass loading ratio for nanocarriers reported to date, preserve and protect the nucleic acid integrity from degradation, and facilitate spontaneous nucleic acid translocation across the plant plasma membrane and cell wall, resulting in transgene expression in the plant cell in vitro and in vivo.

In certain embodiments, the cationic polymer is conjugated to the virus through an amide bond. For example, in certain embodiments, the cationic polymer comprises primary amine group(s) that could react with free carboxy group of certain amino acid residues on a virus coat protein to form amide bond.

In certain embodiments, the cationic polymer is poly-(allylamine) having structure of

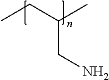

or salt thereof, such as poly-(allylamine) hydrochloride (PAH).

In certain embodiments, poly(allylamine) or salt thereof has a molecular weight of at least about 3 kDa (e.g., 5 kDa, 10 kDa, 15 kDa, 17 kDa, 20 kDa, 30 kDa, 40 kDa, or 50 kDa). In certain embodiments, poly(allylamine) or salt thereof has a molecular weight of about 15-50 kDa. In certain embodiments, poly(allylamine) or salt thereof has a molecular weight of about 17.5 kDa. The molecular weight of PA polymer could be determined by Gel permeation chromatography (GPC).

In certain embodiments, the poly-(allylamine) or salt thereof has a molecular weight of at least about 10 kDa. In certain embodiments, the poly-(allylamine) or salt thereof has a molecular weight of about 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150 kDa or higher. In certain embodiments, the poly-(allylamine) or salt thereof has a molecular weight of at least about 15 kDa. In certain embodiments, the poly-(allylamine) or salt thereof has a molecular weight of at least about 17.5 kDa. In certain embodiments, the poly-(allylamine) or salt thereof has a molecular weight of at least about 50 kDa. In certain embodiments, the poly-(allylamine) or salt thereof has a molecular weight of at least about 150 kDa.

In certain embodiments, the poly-(allylamine) or salt thereof has a molecular weight of about 10-150, 10-50, 10-20, 15-150, 15-50, 17-150, or 10-18 kDa. In certain embodiments, the poly-(allylamine) or salt thereof has a molecular weight of about 10-20 kDa. In certain embodiments, the poly-(allylamine) or salt thereof has a molecular weight of about 10-50 kDa. In certain embodiments, the poly-(allylamine) or salt thereof has a molecular weight of about 15-20 kDa. In certain embodiments, the poly-(allylamine) or salt thereof has a molecular weight of about 15-50 kDa.

In certain embodiments, the nanoparticle has an aspect ratio (length over width ratio) that is greater than 3 (e.g., 4, 5, 6, 7, 8, 9, 10, 11, 12, 13, 14, 15, or 16). In certain embodiments, the nanoparticle has an aspect ratio (length over width ratio) that is about 6-16.

In certain embodiments, the nanoparticle has an aspect ratio that is greater than 4, 5, 6, 7, 8, 9, 10, 11, 12, 13, 14, 15 or 16. In certain embodiments, the nanoparticle has an aspect ratio that is about 3-17, 4-17, 5-17, 6-16, 3-12, 5-15, or 4-10.

In certain embodiments, the nanoparticle has a length of about 60-600 nm or 60-300 nm (e.g., 100-300 nm, 150-300 nm, or 200-300 nm). In certain embodiments, the nanoparticle has a length of about 60, 70, 80, 90, 100, 120, 140, 160, 180, 200, 220, 240, 260, 280, or 300 nm.

In certain embodiments, the nanoparticle has a length of about 60-600, 80-500, 100-600, 100-500, 90-400, or 70-450 nm. In certain embodiments, the nanoparticle has a length of about 60, 70, 80, 90, 100, 120, 140, 160, 180, 200, 220, 240, 260, 280, 300, 350, 400, 450, 500, 550, or 600 nm.

In certain embodiments, the nanoparticle has a length of about 60-200 nm. In certain embodiments, the nanoparticle has a length of about 80-140 nm or 70-190 nm.

In certain embodiments, the nanoparticle has a width of about 15-300 nm or 15-30 nm (e.g., 18-25 nm, 18-23 nm, or 18-20 nm).

In certain embodiments, the nanoparticle has a hydrodynamic diameter (as determined by DLS) of at least about 300 nm (e.g., 310, 320, 330, 340, 350, 360 nm). In certain embodiments, the nanoparticle has a hydrodynamic diameter (as determined by DLS) of about 300-1000 nm, 300-800 nm, 300-600 nm, or 300-400 nm.

In certain embodiments, the nanoparticle has a hydrodynamic diameter (as determined by DLS) of at least about 200 nm (e.g., 250, 260, 270, 280, or 290 nm). In certain embodiments, the nanoparticle has a hydrodynamic diameter (as determined by DLS) of about 200-1000 nm, 230-800 nm, 260-600 nm, 260-380 nm or 270-400 nm.

In certain embodiments, the nanoparticle has a hydrodynamic diameter (as determined by DLS) of at least about 100 nm (e.g., 120, 160, 180, or 190 nm). In certain embodiments, the nanoparticle has a hydrodynamic diameter (as determined by DLS) of about 100-1000 nm, 110-800 nm, 120-600 nm, 160-380 nm or 180-400 nm.

In certain embodiments, the nanoparticle is a live virus. In certain embodiments, the nanoparticle is an inactivated virus (e.g., inactivated by UV light). In certain embodiments, the nanoparticle is selected from the group consisting of tobacco mosaic virus, tobacco mild green mosaic virus, cowpea mosaic virus, and potato virus X. In certain embodiments, the nanoparticle is a live or inactivated tobacco mosaic virus. In certain embodiments, the nanoparticle is a live or inactivated cowpea mosaic virus. In certain embodiments, the nanoparticle is a live or inactivated potato virus X. In certain embodiments, the nanoparticle is a live or inactivated tobacco mild green mosaic virus (TMGMV).

In certain embodiments, prior to loading nucleic acid cargo, the nanoparticle has a zeta potential of about 40-65 mV or 50-65 mV. In certain embodiments, prior to loading nucleic acid cargo, the nanoparticle has a zeta potential of about 40-60 mV or 55-65 mV.

In certain embodiments, prior to loading nucleic acid cargo, the nanoparticle has a zeta potential of about 40, 45, 50, 55, 60, or 65 mV.

The conjugated cationic polymer such as poly(allylamine) is capable of conferring a positive charge to the nanoparticle outer surface, producing a cationic nanoparticle suitable for binding nucleic acid (e.g., DNA, including ssDNA, or dsDNA such as plasmid DNA; or RNA such as siRNA, etc.).

In certain embodiments, the nucleic acid is DNA. In certain embodiments, the nucleic acid is a single-stranded DNA (ssDNA). In certain embodiments, the nucleic acid is double-stranded (dsDNA). In certain embodiments, the nucleic acid is plasmid DNA.

In certain embodiments, the nucleic acid is RNA. In certain embodiments, the nucleic acid is siRNA. In certain embodiments, the nucleic acid is mRNA.

Accordingly, in certain embodiments, nucleic acid, e.g., DNA, is adsorbed on the outer surface of nanoparticle(s) described herein (e.g., live and/or inactivated, tobacco mosaic virus, tobacco mild green mosaic virus, cowpea mosaic virus, potato virus X, or a combination thereof, which is conjugated with the cationic polymer, such as PAH).

As used herein, the term “adsorbed” or “loaded” refers to the attachment of nucleic acid on the outer surface of the nanoparticle through electrostatic interactions.

In certain embodiments, the nucleic acid (e.g., DNA) is linked to a detectable agent (e.g., a fluorophore).

In certain embodiments, the mass ratio of nanoparticle to nucleic acid (e.g., DNA or RNA) is about 100:1 to 1:12. In certain embodiments, the mass ratio of nanoparticle to nucleic acid is about 1:1 to 1:12. In certain embodiments, the mass ratio of nanoparticle to nucleic acid is about 1:3 to 1:6 (e.g., 1:4, or 1:5). In certain embodiments, the mass ratio of nanoparticle to nucleic acid is about 10:1, 9:1, 8:1, 7:1, 6:1 or 5:1.

In certain embodiments, the mass ratio of nanoparticle to nucleic acid is about 10:1 to 1:20. In certain embodiments, the mass ratio of nanoparticle to nucleic acid is about 10:1 to 1:12. In certain embodiments, the mass ratio of nanoparticle to nucleic acid is about 1:1 to 1:20. In certain embodiments, the mass ratio of nanoparticle to nucleic acid is about 1:1, 1:2, 1:3, 1:4, 1:5, 1:6, 1:7, 1:8, 1:9, 1:10, 1:11, or 1:12.

In certain embodiments, after loading nucleic acid cargo, the nanoparticle may have a zeta potential of about 10-60 mV, 20-55 mV, 30-53 mV, 35-51 mV, or 40-50 mV.

In certain embodiments, after loading nucleic acid cargo, the nanoparticle may have a zeta potential of about 10, 15, 20, 25, 30, 35, 40, 45, 50, 55, or 60 mV.

In certain embodiments, after loading nucleic acid cargo, the nanoparticle may have a zeta potential of about 30-60 mV or 35-60 mV.

In certain embodiments, after loading nucleic acid cargo, the nanoparticle may have a zeta potential of about 35-50 mV, 37-47 mV, or 36-48 mV.

In certain embodiments, after loading nucleic acid cargo, the nanoparticle may have a zeta potential of about 58-59 mV, 55-60 mV, 50-60 mV, or 45-60 mV.

In certain embodiments, the DNA adsorbed on the nanoparticle is protected from enzymatic degradation. In certain embodiments, the DNA adsorbed on the nanoparticle is protected from degradation by DNase I nuclease.

For illustration purpose, as a non-limiting example, some embodiments of the invention provide an engineered Tobacco mild green mosaic virus (TMGMV) nanoparticle that has a cationic surface suitable for adsorption of nucleic acid(s) through electrostatic interactions. For example, described herein include a nanoparticle comprising a Tobacco mild green mosaic virus (TMGMV) coat protein (CP) that is conjugated with poly(allylamine) or salt thereof (e.g., poly-(allylamine) hydrochloride (PAH)).

In certain embodiments, the Tobacco mild green mosaic virus (TMGMV) coat protein (CP) may have a molecular weight of about 17.5 kDa. In certain embodiments, the TMGMV CP may comprise, or consist of, an amino acid sequence of about 158 amino acids in length (e.g., see the last 158 amino acids (starting with proline) of NCBI Accession Number QIC50208, AAD41792, UNI81575, or CAK18173). In certain embodiments, the TMGMV CP comprises amino acid residue(s) that comprises carboxyl (e.g., as part of amino acid side chain group) that could form an amide bond with an amino group of poly(allylamine) or salt thereof, as shown herein to form CP-PA conjugate.

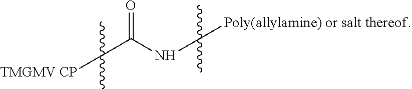

As used herein, the term “conjugate” refers to two or more elements that are covalently linked, for example, a viral protein (e.g., TMGMV coat protein) may be covalently linked with a cationic polymer, such as poly(allylamine) via amide bond (—C(═O)NH—).

In certain embodiments, the nanoparticle (e.g., TMGMV nanoparticle) comprises, or consist of, 2130 copies of the coat protein (CP), wherein one or more copies of the coat protein (CP) is conjugated with poly(allylamine) or salt thereof.

In certain embodiments, the nanoparticle (e.g., TMGMV or iTMGMV nanoparticle) comprises, or consist of, at least 200, 300, 400, 500, 600, 700, 800, 900, 1000, 1100, 1200, 1300, 1400, 1500, 1600, 1700, 1800, 1900, 2000, or 2100 copies of the coat protein (CP), wherein one or more copies of the coat protein (CP) is conjugated with poly(allylamine) or salt thereof.

In certain embodiments, the poly-(allylamine) conjugated coat protein has a molecular weight of at least about 30 kDa (e.g., 31 kDa, 32 kDa, 33 kDa, 34 kDa, or higher). In certain embodiments, the poly-(allylamine) conjugated coat protein has a molecular weight of at least about 35 kDa.

### Compositions and Methods

Certain embodiments of the invention provide a composition comprising one or more nanoparticle(s) described herein, and a carrier.

In certain embodiments, the one or more nanoparticle(s) is a live virus, inactivated virus, or combination thereof. In certain embodiments, the one or more nanoparticle(s) is selected from the group consisting of tobacco mosaic virus, tobacco mild green mosaic virus, cowpea mosaic virus, potato virus X, or combination thereof.

In certain embodiments, the composition is in a liquid form. In certain embodiments, the carrier is an aqueous solution. In certain embodiments, the carrier is a buffered solution (e.g., saline or PBS).

In certain embodiments, the composition is in a solid form, for example, the composition may be in a lyophilized form which may contain carrier such as bulking agent (e.g., mannitol or glycine) and cryoprotectant/lyoprotectant (e.g., trehalose or sucrose). Lyophilized composition can be reconstituted into a liquid form using, e.g., saline or water before use.

Certain embodiments of the invention provide a method of making nanoparticle(s) or a composition as described herein, comprising conjugating a cationic polymer to nanoparticle(s), wherein the nanoparticle is a live or inactivated virus. In certain embodiments, the nanoparticle(s) is selected from the group consisting of tobacco mosaic virus, tobacco mild green mosaic virus, cowpea mosaic virus, potato virus X, or combination thereof. In certain embodiments, the nanoparticle is a nanoparticle as described herein.

In certain embodiments, the conjugating comprises forming an amide bond between the cationic polymer and viral protein (e.g., virus coat protein) comprised in the nanoparticle.

In certain embodiments, the conjugation is conducted via a carbodiimide reaction such as through NHS-EDC reaction.

In certain embodiments, the method further comprises contacting the nanoparticle with a nucleic acid (e.g., DNA or RNA).

In certain embodiments, the cationic nanoparticle is contacted with the nucleic acid at a nanoparticle to nucleic acid mass ratio as described herein.

Certain embodiments of the invention provide a method for delivering nucleic acid to a plant cell, comprising contacting the plant cell with a nanoparticle as described herein. In certain embodiments, the plant cell is contacted in vivo. In certain embodiments, the plant cell is contacted in vitro. In certain embodiments, the nanoparticle is a live virus described herein. In certain embodiments, the nanoparticle is an inactivated virus described herein.

In certain embodiments, the plant cell is a leaf cell. In certain embodiments, the plant cell is a leaf epidermal cell.

In certain embodiments, the plant cell is a protoplast cell.

In certain embodiments, the nucleic acid is delivered into the nucleus of the plant cell.

In certain embodiments, the plant cell is contacted with nanoparticle (comprising nucleic acid cargo) at a concentration of about 0.01 mg/mL to 1 mg/mL (e.g., 0.05 to 0.8 mg/mL, or 0.06 to 0.6 mg/mL or 0.1 to 0.5 mg/mL).

In certain embodiments, the plant cell is contacted with nanoparticle (comprising nucleic acid cargo) at a concentration of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 mg/mL.

In certain embodiments, the nanoparticle (excluding weight of nucleic acid cargo) has a concentration of about 0.01 mg/mL to 0.3 mg/mL (e.g., 0.05 to 0.3 mg/mL, or 0.06 to 0.2 mg/mL or 0.1 to 0.15 mg/mL). In certain embodiments, the nanoparticle (excluding weight of nucleic acid cargo) has a concentration of about 0.01, 0.02, 0.03, 0.04, 0.05, 0.06, 0.07, 0.08, 0.09, 0.1, 0.11, 0.12, 0.13, 0.14, 0.15, 0.2, or 0.3 mg/mL.

In certain embodiments, the nanoparticle (excluding weight of nucleic acid cargo) has a concentration of about 0.1 mg/mL. In certain embodiments, the plant cell is contacted with nanoparticle (comprising nucleic acid cargo) at a concentration of about 0.2, 03, 0.4, 0.5, 0.6, 0.7, or 0.8 mg/mL. In certain embodiments, the mass ratio of nanoparticle and the loaded nucleic acid cargo may be about 1:1, 1:2, 1:3, 1:4, 1:5, 1:6, or 1:7.

In certain embodiments, the nanoparticle (excluding weight of nucleic acid cargo) has a concentration of about 0.05 mg/mL. In certain embodiments, the plant cell is contacted with nanoparticle (comprising nucleic acid cargo) at a concentration of about 0.1, 0.15, 0.2, 0.25, 0.3, 0.35, or 0.4 mg/mL. In certain embodiments, the mass ratio of nanoparticle and the loaded nucleic acid cargo may be about 1:1, 1:2, 1:3, 1:4, 1:5, 1:6, or 1:7.

In certain embodiments, the plant cell is contacted with nanoparticle (comprising nucleic acid cargo) at a concentration of about 0.01 mg/mL to 1 mg/mL (e.g., 0.02 to 0.9 mg/mL, 0.05 to 0.8 mg/mL, 0.1 to 0.7 mg/mL, 0.1 to 0.6 mg/mL, 0.1 to 0.5 mg/mL, 0.1 to 0.4 mg/mL, 0.1 to 0.3 mg/mL, 0.2 to 0.8 mg/mL, or 0.1 to 0.2 mg/mL).

In certain embodiments, the nucleic acid cargo has a weight of about 0.1 to 30 g (e.g., 0.1, 0.2, 0.3, 0.4, 0.5, 1, 2, 3, 4, 5, 6, 7, 8, 9, 10, 15, 20, or 25 μg). In certain embodiments, the nucleic acid cargo (e.g., plasmid DNA) has a weight of about 5-30, 5-25, 10-30, 10-25, 15-25, or 15-30 μg.

In certain embodiments, the viability of plant cells after contacting the nanoparticle loaded with nucleic acid (e.g., plasmid DNA) is at least about 30%, 35%, 40%, 45%, 50%, 60%, 65%, 70%, 75%, 80%, 85%, 90%, 95% or higher. In certain embodiments, the viability of plant cells after contacting the nanoparticle loaded with nucleic acid (e.g., plasmid DNA) is at least about 60%, 65%, or higher. In certain embodiments, the viability of plant cells after contacting the nanoparticle loaded with nucleic acid (e.g., plasmid DNA) is at least about 80%, 85%, or higher.

In certain embodiments, the viability reduction of plant cells after contacting the nanoparticle loaded with nucleic acid (e.g., plasmid DNA) is no more than about 30%, 25%, 20%, 15%, 10%, or 5% as compared to a control (e.g., untreated plant cells or plant cells treated with vehicle control).

In certain embodiments, the plant cell is contacted at about 4-35° C., 4-30° C., or 4-25° C. In certain embodiments, the plant cell is contacted at about 4° C. In certain embodiments, the plant cell is contacted at about 25° C. In certain embodiments, the plant cell is contacted at about 10, 15, or 20° C.

Certain exemplary embodiments of the invention include:

Embodiment 1. A composition as described herein.

Embodiment 2. A nanoparticle as described herein.

Embodiment 3. A nanoparticle, comprising a cationic polymer (e.g., PAH) conjugated to the nanoparticle, wherein the nanoparticle is a live virus, an inactivated virus, or a combination thereof.

Embodiment 4. The nanoparticle of Embodiment 3, wherein the nanoparticle is selected from the group consisting of tobacco mosaic virus, tobacco mild green mosaic virus, cowpea mosaic virus, potato virus X or a combination thereof, and wherein the cationic polymer is PAH.

Embodiment 5. A composition comprising a nanoparticle, wherein the nanoparticle (e.g., a live virus, inactivated virus, or a combination thereof) is selected from the group consisting of tobacco mosaic virus, tobacco mild green mosaic virus, cowpea mosaic virus, potato virus X, or a combination thereof, and wherein the nanoparticle is conjugated with a cationic polymer (e.g., PAH).

Embodiment 6. A nanoparticle, comprising a Tobacco mild green mosaic virus (TMGMV) coat protein (CP) that is conjugated with poly-(allylamine) or salt thereof (e.g., PAH).

Embodiment 7. The nanoparticle of any one of Embodiment 4 or 6, wherein the poly-(allylamine) has a molecular weight of at least about 10 kDa (e.g., 17.5 kDa).

Embodiment 8. The nanoparticle of any one of Embodiments 6-7, wherein the poly-(allylamine) conjugated coat protein has a molecular weight of at least about 30 kDa (e.g., 35 kDa).

Embodiment 9. The nanoparticle of any one of Embodiments 3-4 and 6-8, wherein the nanoparticle has an aspect ratio that is greater than 3 (e.g., 10 or 16).

Embodiment 10. The nanoparticle of any one of Embodiments 3-4 and 6-9, wherein the nanoparticle has a length of about 60-600 nm.

Embodiment 11. The nanoparticle of any one of Embodiments 3-4 and 6-10, wherein the nanoparticle has a width of about 15-300 nm (e.g., 18-25 nm).

Embodiment 12. The nanoparticle of any one of Embodiments 3-4 and 6-11, wherein the nanoparticle has a Zeta potential of about 50-65 mV.

Embodiment 13. The nanoparticle of any one of Embodiments 3-4 and 6-12, wherein nucleic acid (e.g., DNA) is adsorbed on the nanoparticle outer surface.

Embodiment 14. The nanoparticle of Embodiment 13, wherein the nucleic acid is plasmid DNA or ssDNA.

Embodiment 15. The nanoparticle of Embodiment 13, wherein the nucleic acid is RNA.

Embodiment 16. The nanoparticle of any one of Embodiments 13-15, wherein the nanoparticle has a Zeta potential of about 10-60 mV (e.g., 30-53 mV).

Embodiment 17. The nanoparticle of any one of Embodiments 13-16, wherein the mass ratio of nanoparticle to nucleic acid (e.g., DNA) is about 10:1 to 1:12.

Embodiment 18. The nanoparticle of any one of Embodiments 13-17, wherein the mass ratio of nanoparticle to nucleic acid (e.g., DNA) is about 1:3 to 1:6.

Embodiment 19. The nanoparticle of Embodiment 14, wherein the DNA is protected from degradation by DNase I nuclease.

Embodiment 20. A method as described herein.

Embodiment 21. A method for delivering nucleic acid to a plant cell, comprising contacting the plant cell with a nanoparticle as described herein, e.g., a nanoparticle according to any one of Embodiments 13-19.

Certain non-limiting embodiments are further illustrated by the Examples below:

### Example 1. Plasmid DNA Delivery by Virus Like Nanocarriers in Plant Cells

Tobacco mild green mosaic virus (TMGMV) like nanocarriers were designed for gene delivery to plant cells. TMGMVs were coated with a polycationic biopolymer, poly-(allylamine) hydrochloride (PAH) to generate highly charged nanomaterials (PAH-TMGMV) (56.20±4.7 mV) that efficiently load and deliver single-stranded DNA and plasmid DNA to plant cells. The PAH-TMGMV (also referred to as TMGMV-PAH in Examples 1-2) were uptaken through spontaneous and energy-independent mechanisms in isolated mesophyll protoplasts collected from Arabidopsis thaliana. PAH-TMGMV nanocarriers delivered DNA into the nucleus with 11% colocalization efficiency determined by confocal microscopy. The PAH-TMGMV also effectively delivered a plasmid DNA encoding a green fluorescent protein (GFP) to the protoplast nucleus, evidenced by GFP expression using confocal microscopy and biochemical analysis. More than 70% of protoplasts remained viable after DNA delivery mediated by PAH-TMGMV. Virus-like nanocarrier mediated gene delivery can act as a facile and biocompatible tool for advancing genetic engineering in plants.

### INTRODUCTION

The rapid increase of the human global population is projected to require a 60% increase in food production by 2050.^{1,2} Plant genetic engineering has been widely employed to generate crops with increased yield,² improved quality, enhanced resistance to herbicides,³ insects,⁴ diseases,^{5,6} biotic and abiotic stresses.^{7,8} Genetically modified plants for biomanufacturing also hold immense potential for synthesizing small-molecule drugs,⁹ recombinant protein therapeutics,^{10,11} and vaccines.^{12,13} Despite numerous biotechnological advancements over the past decades, the genetic transformation of many plant species still poses considerable challenges. The delivery of transgenes into plant species mainly relies on two transformation methods: Agrobacterium tumefaciens-mediated transformation system¹⁴ and particle bombardment.¹⁵ However, the Agrobacterium-mediated system has some significant drawbacks such as uncontrollable target gene integration into the host chromosomes causing positional effects on gene expression, and many monocotyledonous plant species are inherently resistant to Agrobacterium infection.¹⁶ Biolistics has been utilized in various plant species, as a random gene delivery system into the host nucleus, mitochondria, and chloroplast.³ Particle bombardment is performed by high-pressure gene gun delivery that damages host genomic DNA and results in random insertions of multiple copies of the gene with a very high frequency,¹⁷ which has constrained its applications. The particle bombardment system is also expensive, requires labor intensive tissue culture and selection, has low transformation efficiency, and has not been successfully implemented in diverse plant species.¹⁸ Therefore, there is a pressing need for a versatile, plant species independent, and easy to use tool for plant genetic transformation, allowing for efficient delivery of exogenous genes.

Recent advancements in nanotechnology have unveiled the potential of nanomaterials in facilitating the delivery of genetic materials such as plasmid DNA,¹⁹⁻²¹ and siRNA,^{22,23} as well as biomacromolecules like functional proteins,²⁴ active ingredients,^{25,26} nutrients,²⁷ and therapeutics²⁸ in plants. The single-walled carbon nanotubes (SWCNTs),^{19,20,29} mesoporous silica nanoparticles (MSNs),^{30,31} layered double hydroxide (LDH) clay nanosheets,²² and functional peptide-DNA complexes^{21,32} have demonstrated uptake into plant cells without mechanical assistance to deliver functional DNA/RNA cargoes. Several studies have demonstrated the possibility of carbon nanotube mediated gene delivery in plant nucleus,^{19,32} chloroplast,^{20,29} and mitochondrial²¹ genome. However, there is a need to develop high aspect ratio nanomaterials for plant transformation that are degradable, biocompatible, and manufactured with controlled aspect ratios at large scale. We turned toward plant virus nanoparticles as a biodegradable, cost-effective, and easily scalable nanotechnology with tunable surface chemistry.^{25,26,33}.

Tobacco mild green mosaic virus (TMGMV)³⁴ is a plant virus within the tobamovirus genus, also known as the U2 strain of tobacco mosaic virus (TMV), approved by the U.S. Environmental Protection Agency (EPA) for use in bioherbicides.³⁵ The nucleoprotein components of TMGMV are self-assembled from 2130 identical copies of a coat protein and ssRNA to form a 300×18 nm soft matter rod-shaped structure with a 4 nm wide hollow interior channel.^{25,34,36} The nanoparticles derived from TMGMV have unique physio-chemical properties such as biodegradability (protein-based particles), the ability to self-assemble into identical and high aspect ratio structures, and large scale economical production with high purity and reproducibly.^{25,37} TMGMV can be functionalized with cargo through covalent chemistry³⁸ or encapsulation.²⁵ TMGMV particles have been utilized as a carrier for active ingredients such as a porphyrin-based photosensitizer drugs (Zn-Por) for cancer cell abolition of melanoma and cervical cancer models,³⁶ as well as ivermectin to treat plants infected with parasitic nematodes.^{25,26} Plant virus derived vectors (plasmids with virus genetic elements) have been extensively used for genetic engineering in plants through the mechanical inoculation of plasmid DNA, biolistics, vascular puncture, agroinoculation, or insect mediated vector delivery.^{39,40} Virus applications have also focused on the delivery of RNA packaged inside the capsid for modifying gene expression.⁴¹ Herein we propose using biocompatible, degradable and engineered TMGMVs with controlled manufacturing properties as a carrier for facile delivery of plasmid DNA without mechanical or biological aid.

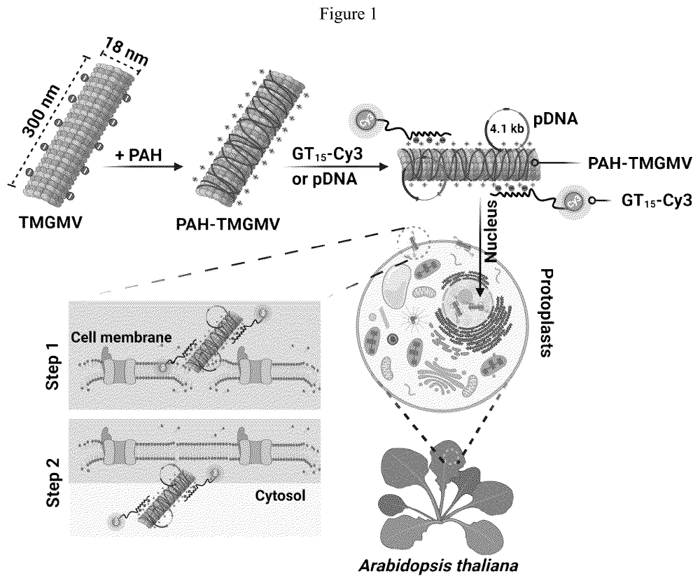

In this Example, we developed TMGMV-based nanomaterials as a platform for the nuclear delivery of DNA in Arabidopsis thaliana mesophyll protoplasts (FIG. 1). We functionalized TMGMV by covalently coating a polycationic biopolymer, poly-(allylamine) hydrochloride (PAH), on the TMGMV surface (PAH-TMGMV). The PAH imparts a positive charge to PAH-TMGMV for binding to DNA through electrostatic interactions. To determine whether PAH-TMGMV delivered single stranded DNA (ssDNA) into protoplast cells without using mechanical aid while maintaining biocompatibility, we employed confocal microscopy to track the ssDNA cargo covalently bonded to a fluorophore (Cy3) and protoplast bioavailability assays. We also demonstrated the high loading capacity of plasmid DNA (pDNA) onto the PAH-TMGMV, and assessed pDNA delivery, uptake mechanism, and transgene expression in protoplasts. This Example demonstrates gene delivery mediated by PAH-TMGMV nanocarriers. Using TMGMVs for DNA delivery in plant cells offers a promising solution for plant genetic transformations that is scalable and biocompatible with high manufacturing quality and reproducibility.

### Results and Discussion

### Virus Like Nanocarriers for DNA Delivery

The selection of polymer coating for TMGMV focused on cationic biopolymers capable of binding electrostatically with negatively charged pDNA. Among various options, PAH, polylysine, and polyarginine were prioritized due to their higher pKa values (above pH 8) and FDA approval for other applications. TMGMV coated with polylysine and polyarginine were negatively charged, making them likely unsuitable for pDNA coating (FIG. 20). In contrast, TMGMV-PAH were positively charged, and therefore, PAH was chosen as the coating for TMGMV in this Example.

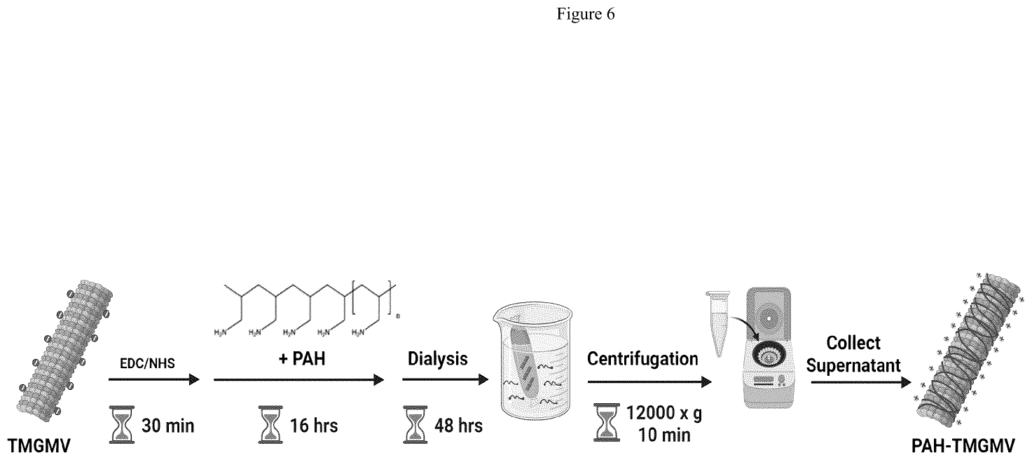

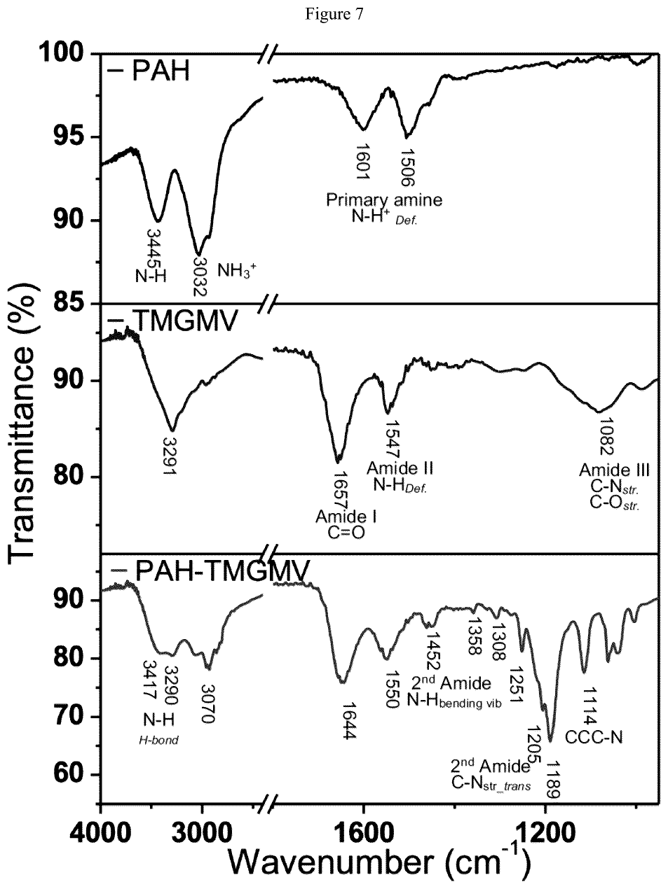

We designed cationic TMGMV by covalent conjugation with a polycationic biopolymer, PAH, capable of binding through electrostatics with negatively charged DNA. PAH has been extensively used for pharmaceutical and drug delivery applications due to its high water-solubility and biodegradable properties.^{42,43} We utilized a carbodiimide coupling reaction to covalently bond the amine functional groups of PAH to the carboxyl groups in TMGMV (FIG. 6)³⁸ and the chemical conjugation was confirmed by Fourier-transform infrared spectroscopy (FTIR) (FIG. 7). To investigate DNA delivery by PAH-TMGMV in protoplasts, we used confocal microscopy to track ssDNA oligonucleotides (GT)₁₅ covalently linked to the Cy3 fluorescent dye (GT₁₅.Cy3). Cy3 is bright, photostable, and its emission range does not overlap with chloroplast autofluorescence. GT₁₅-Cy3 has been previously employed for coating positively charged carbon nanotubes for determining subcellular localization in plants.^{20,29,44}

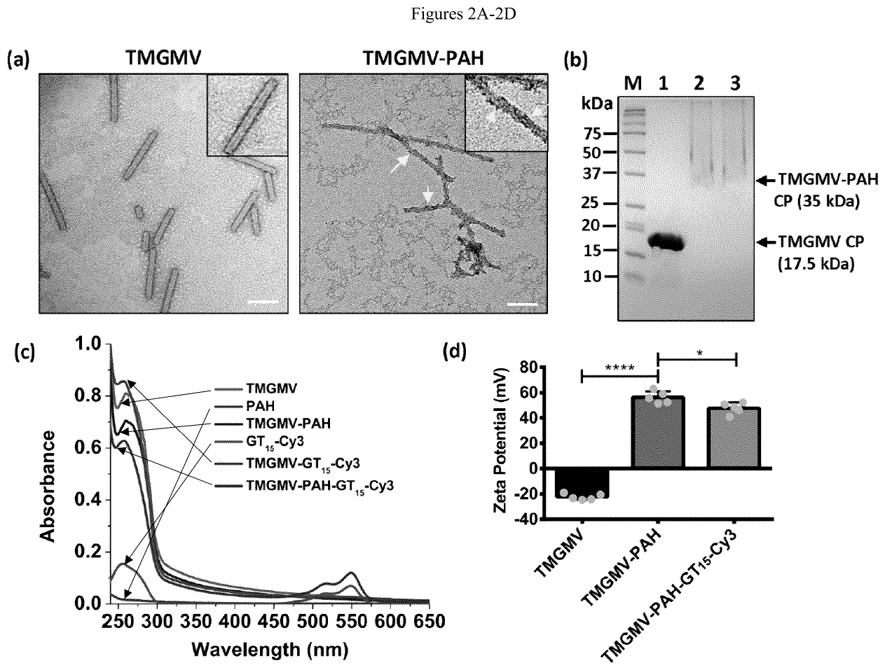

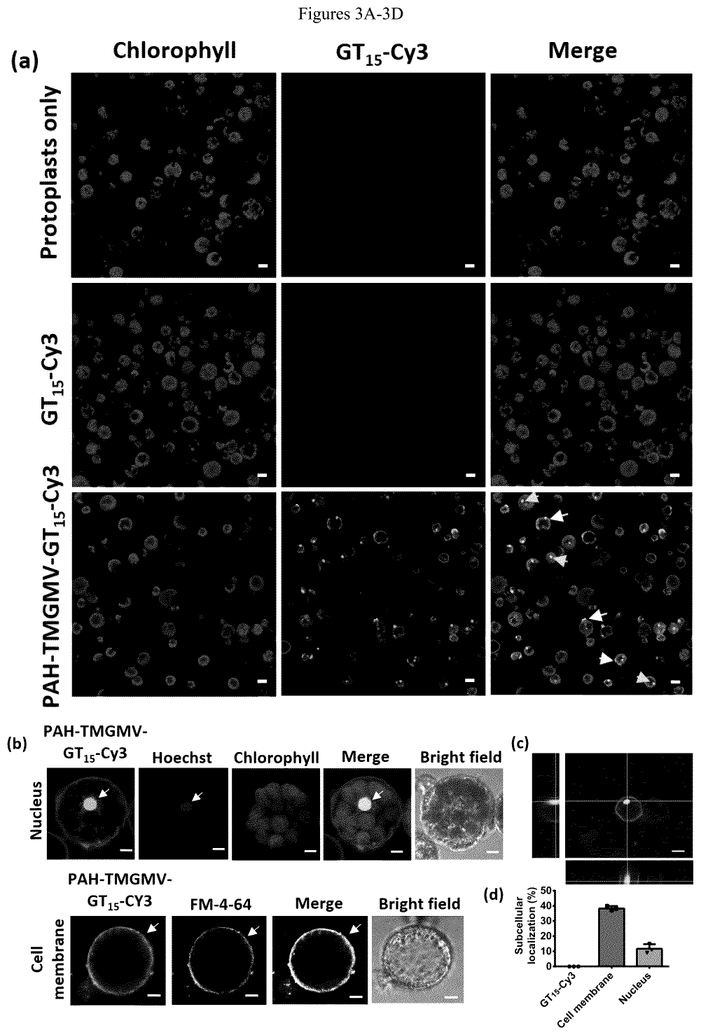

We characterized TMGMV, PAH-TMGMV and GT₁₅.Cy3 loaded PAH-TMGMV (PAH-TMGMV-GT₁₅.Cy3) by UV-Vis, dynamic light scattering (DLS), Zeta potential (ζ), transmission electron microscopy (TEM), Nu-PAGE, and fluorescence emission spectra. TEM imaging of TMGMV and PAH-TMGMV shows high aspect ratio, rod-shaped nanostructures (FIG. 2A) consistent with previous studies using TMGMV for pesticide delivery.^{25,38} The PAH-TMGMV had a rough surface not observed in TMGMV (FIG. 2A), indicating coating of the PAH polymer on the TMGMV surface. Based on TEM analysis, the average length of TMGMV and PAH-TMGMV was non-significantly different, 129.9±57.7 nm, and 191.3±95 nm, respectively. Notably, broken nanomaterials were also observed in both uncoated TMGMV and PAH-TMGMV, which can occur during preparation or imaging of the TMGMV TEM samples.^{25,38} Furthermore, the conjugation of PAH (˜17.5 kDa) to TMGMV coat protein (CP) was confirmed by denatured Nu-PAGE analysis, which indicated the presence of higher molecular weight bands at ˜35 kDa, in addition to the TMGMV CP band at ˜17.5 kDa monomer (FIG. 2B). The smeared protein bands were observed due to the high positive charge of PAH-TMGMV CP (56.20±4.7 mV) that hinders the relative mobility towards the electrode in the Nu-PAGE system. Both TEM and Nu-PAGE analysis indicate that PAH is coated onto the PAH-TMGMV.

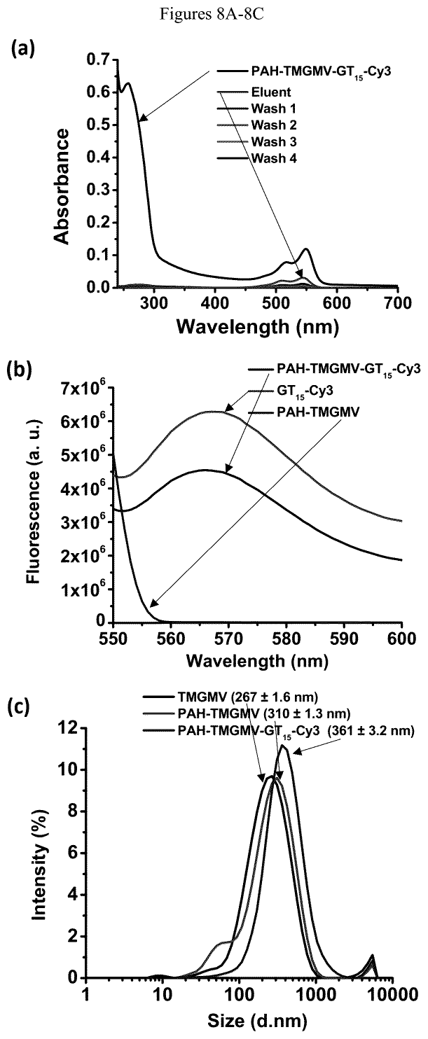

The UV-Vis absorbance spectrum of TMGMV, PAH-TMGMV, and PAH-TMGMV-GT₁₅-Cy3 indicated characteristic absorption peaks at 260 nm (FIG. 2C). PAH-TMGMV-GT₁₅-Cy3 showed distinct absorption peaks at 550 nm that corresponded to the Cy3 dye on the PAH-TMGMV (FIG. 2C). To validate the GT₁₅-Cy3 binding to PAH-TMGMV and confirm the absence of unbound dye, the sample was purified using a centrifugal filter unit (100 K MWCO). Following the second wash step, no absorbance corresponding to GT₁₅-Cy3 was detected in the eluent (FIG. 8A) whereas PAH-TMGMV-GT₁₅-Cy3 exhibited fluorescence emission peaks at 564 nm, attributed to the attachment of GT₁₅.Cy3 on PAH-TMGMV (FIG. 8B). DLS analysis indicated well dispersed nanomaterials with increasing hydrodynamic diameter from 267±1.6 nm for TMGMV to 310±1.3 nm for PAH-TMGMV and 361±3.2 nm for PAH-TMGMV-GT₁₅-Cy3 (P<0.005) (FIG. 8C). We observed a significant change of (potential after conjugation of PAH from negative charged TMGMV (−22.37±2.3 mV) to highly positive charged PAH-TMGMV (56.20±4.7 mV) (P<0.0001) (FIG. 2D), clearly indicating binding of polycationic PAH to the TMGMV surface. As expected, the (potential for PAH-TMGMV slightly decreased from 56.20±4.7 mV to 47.69±4.4 mV when loading GT₁₅-Cy3 (P<0.05) (FIG. 2D) due to the electrostatic bonding between the negatively charged GT₁₅ and the positively charged PAH-TMGMV.

DNA Delivery into Plant Cells

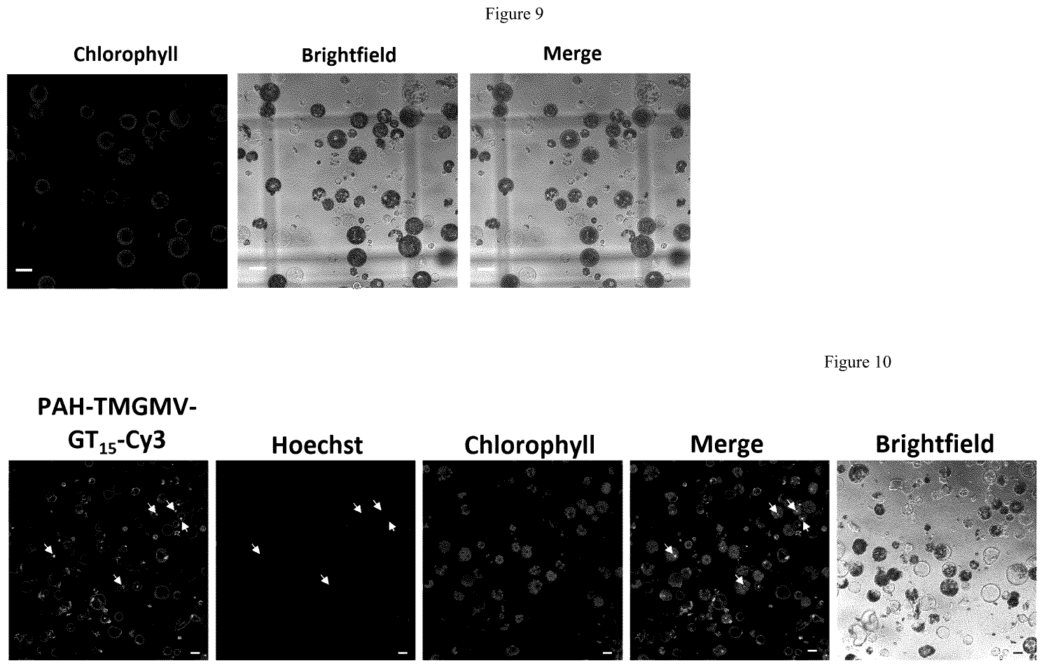

To examine in vitro DNA delivery and subcellular localization in plant cells using PAH-TMGMV as a nanocarrier, Arabidopsis protoplasts were isolated and incubated with PAH-TMGMV coated with GT₁₅-Cy3. Protoplasts are model systems for gene expression analysis that have been used in numerous plant nanoparticle studies of uptake and gene delivery.^{19,29,45} To assess the delivery of GT₁₅-Cy3 bound to PAH-TMGMV and their subcellular localization using confocal microscopy, isolated protoplasts (FIG. 9) were incubated with 0.1 mg/mL of PAH-TMGMV-GT₁₅-Cy3 at room temperature for 2 hours before imaging. Confocal fluorescence microscopy images indicated a significant level of GT₁₅-Cy3 fluorescence signal in protoplast cell membranes, and nuclei when treated with PAH-TMGMV-GT₁₅.Cy3 (FIG. 3A). In contrast, control confocal images of protoplasts treated with GT₁₅-Cy3 did not show GT₁₅-Cy3 fluorescence signal indicating that GT₁₅-Cy3 alone cannot be uptaken by protoplasts (FIG. 3A). To confirm PAH-TMGMV-GT₁₅-Cy3 interaction with protoplast cell membranes and GT₁₅-Cy3 nuclear delivery by PAH-TMGMV, protoplasts were stained with a cell membrane marker FM-4-64 and a nuclear staining marker Hoechst. The GT₁₅-Cy3 fluorescence was observed localized with FM-4-64 and Hoechst fluorescence signals in protoplasts cell membrane and nucleus, respectively (FIG. 3B, FIG. 10). Orthogonal projections from Z-stacks of different planes (x/y, x/z, or y/z) of the confocal microscope images confirmed nuclear uptake of GT₁₅-Cy3 using PAH-TMGMV as shown by the colocalization with Hoechst fluorescence dye (FIG. 3C). Quantitative subcellular localization analysis indicated that approximately 38%±1.5 of the GT₁₅-Cy3 fluorescence signal was observed in protoplast cell membranes while 11%±3.0 localized with the nuclear marker (Hoechst) (FIG. 3D). Together, our results indicate that high aspect ratio and highly positive charged PAH-TMGMV allow penetration through plant cell membranes and facilitate ssDNA delivery (GT₁₅-Cy3) into the nucleus, similar to inorganic high aspect ratio nanomaterials with positive charge.¹⁹

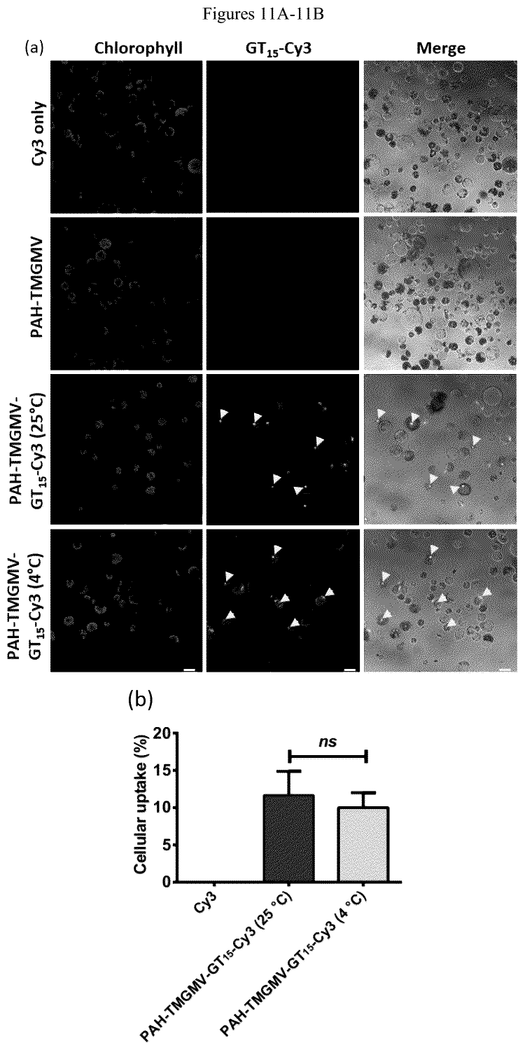

To elucidate the mechanism of DNA delivery into plant cells by PAH-TMGMV, we conducted a cell uptake assay with PAH-TMGMV-GT₁₅-Cy3 at 4° C. to inhibit energy-dependent uptake mechanisms, including endocytosis.⁴⁶ We observed similar percentage of protoplasts with GT₁₅-Cy3 delivery by PAH-TMGMV at 4° C. (10%±1.6) and 25° C. (11%±3.2) (FIG. 11). Thus, DNA delivered by PAH-TMGMV passively traverses the protoplast membrane in an energy-independent mechanism. This is consistent with previous studies demonstrating that highly charged nanomaterials spontaneously penetrate plant cells, by creating temporary pores in their lipid membranes.^{19,20,29,47,48}

### Plasmid DNA Delivery and Expression in Plant Nucleus

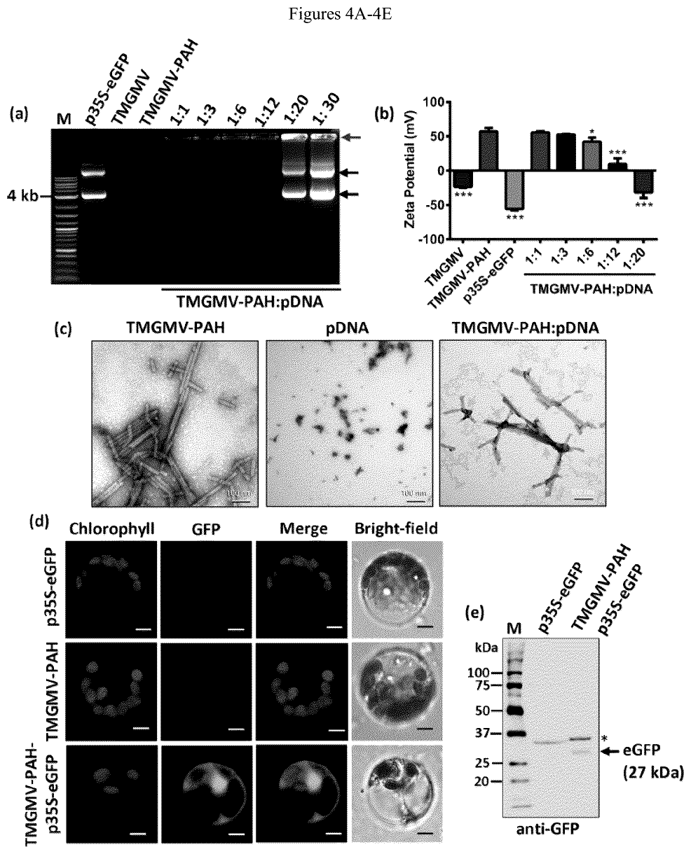

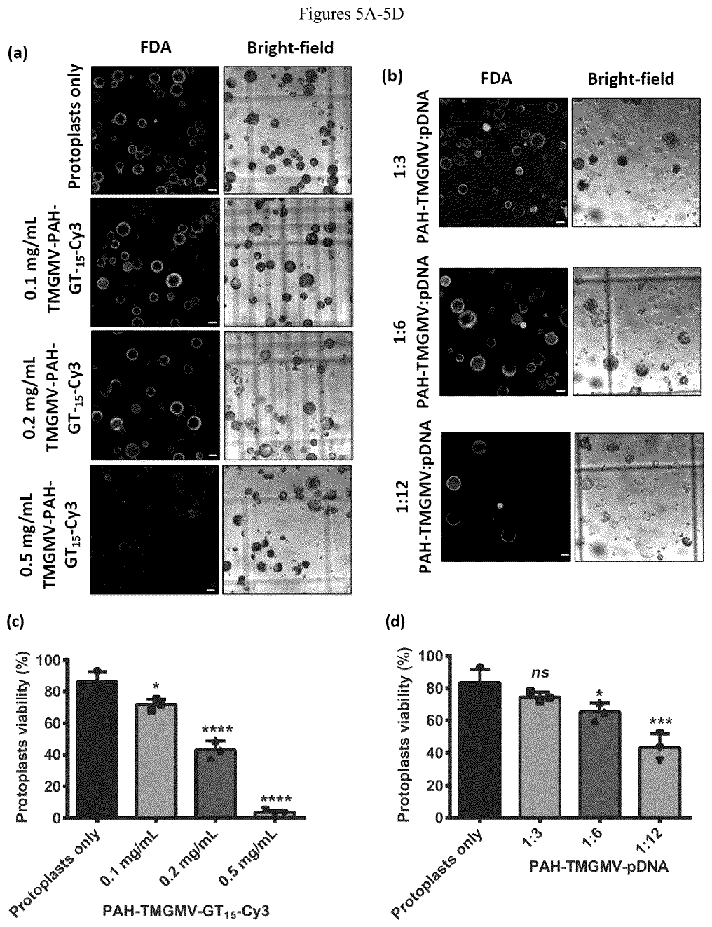

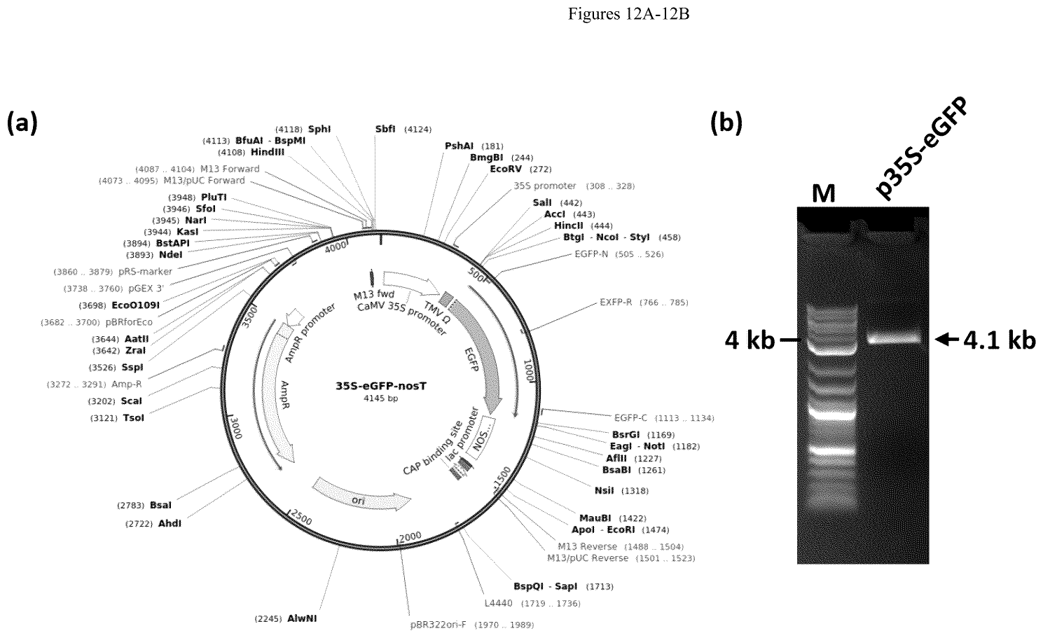

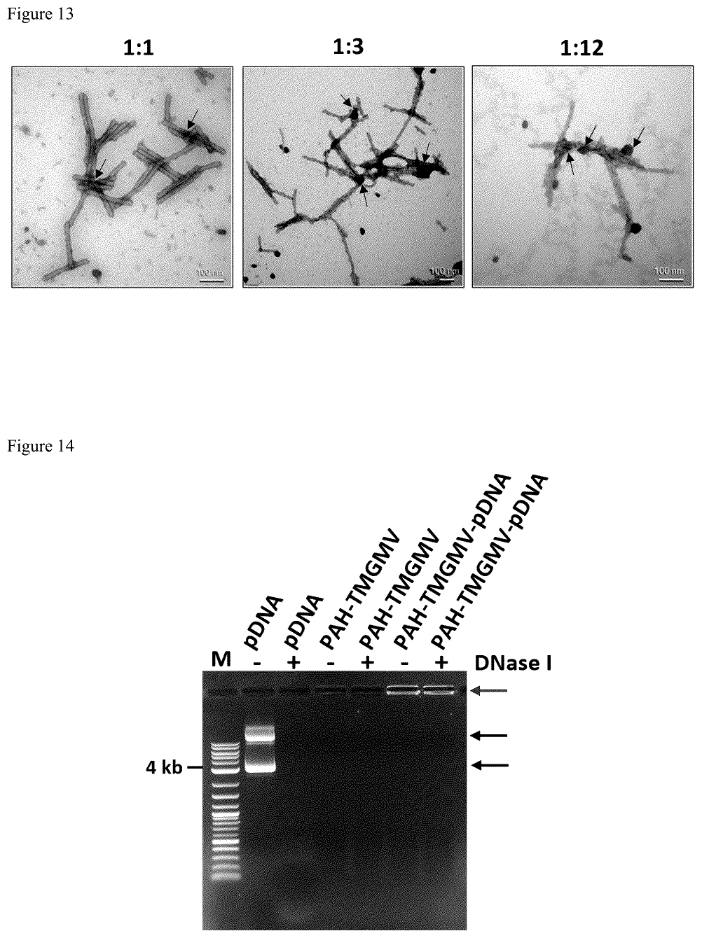

We investigated PAH-TMGMV loading of pDNA, encoding a green fluorescent protein (GFP) in a transient expression vector (p35S-eGFP) (FIG. 12), and delivery in Arabidopsis protoplasts. The PAH-TMGMV-pDNA were loaded at various concentrations of pDNA (PAH-TMGMV:pDNA mass ratios 1:1 to 1:20 w w). The gel electrophoresis of pDNA mobility shift assay (EMSA) showed no unbound or free pDNA running into the agarose gel at a mass ratio of PAH-TMGMV/pDNA=1:1 to 1:12 (w w), meaning that pDNA loading was 100% up to 1:12 (w/w) mass ratio (FIG. 4A). The 1:12 PAH-TMGMV to pDNA mass loading ratio is six times higher than the 1:2 loading of pDNA reported in previous studies using inorganic nanoparticles.¹⁹ Oversaturated and unbound free pDNA bands were observed at PAH-TMGMV:pDNA mass ratios 1:20 (w/w) and higher in EMSA (FIG. 4A). The loading of pDNA gradually reduced ζ potential as the loading ratio of pDNA increased from 1:1 to 1:12 (FIG. 4B) due to the electrostatic bonding between the negatively charged pDNA and the positively charged PAH-TMGMV. The highest decrease in (potential was observed after pDNA loading to PAH-TMGMV at a mass ratio of 1:12, dropping from the initial +57.53±5.2 mV for PAH-TMGMV to +9.57±10.6 mV (FIG. 4B). At the loading mass ratio of 1:20, the (potential became negative −31.17±6.4 mV, representing the oversaturation of the nanocarriers and free pDNA in the suspension. This finding indicates maximum pDNA loading at 1:12 mass ratio and is consistent with our EMSA analysis. We confirmed morphological integrity of PAH-TMGMV loaded with pDNA from 1:1 to 1:12 mass ratios by TEM (FIG. 4C, FIG. 13). In addition, we also assessed pDNA stability by an in vitro pDNA degradation assay using DNase I (nuclease), which showed that pDNA molecules, when loaded onto PAH-TMGMV, were protected from the DNase I nuclease activity (FIG. 14).

To demonstrate pDNA delivery and expression in plant cells, we incubated isolated protoplasts with PAH-TMGMV-pDNA complexes at 1:6 mass ratio having a high positive charge (+42.16±5.1 mV) and loading of pDNA (FIG. 4B) to promote uptake through lipid membranes 45 and increase the amount of pDNA delivery, respectively. We used 25 μg of pDNA for PAH-TMGMV-mediated protoplast transformation, a standard concentration of pDNA (5-30 μg) established for PEG-mediated protoplast transformation.⁴⁹ Therefore, we adjusted the PAH-TMGMV concentration to 0.04 mg/mL to keep a 1:6 mass ratio of pDNA loading. Protoplasts were incubated with PAH-TMGMV-pDNA, and gene expression was determined after 24 hours by confocal fluorescence microscopy imaging. We observed GFP expression in protoplasts when incubated with PAH-TMGMV-pDNA (FIG. 4D). In contrast, protoplasts incubated with pDNA alone and PAH-TMGMV alone did not show GFP expression (FIG. 4D). To determine GFP protein levels, we performed a Western blot analysis on total soluble protein using an anti-GFP antibody, which detected a 27 kDa GFP-specific protein band (FIG. 4E). Together, these analyses show that PAH-TMGMVs have the highest pDNA mass loading ratio for nanocarriers reported to date, preserve and protect the pDNA integrity from degradation, and facilitate pDNA translocation across the plasma membrane without mechanical aid, enabling transgene expression in the nucleus.

Plant Cell Viability after DNA Delivery by PAH-Coated TMGMV

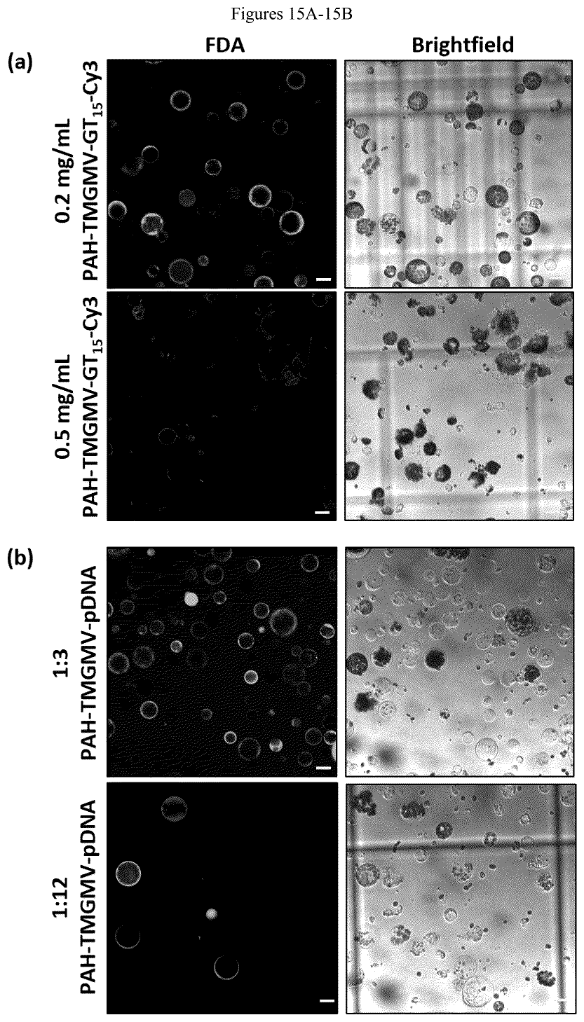

Maintaining cell viability after exposure to nanocarriers with DNA is crucial for enabling biocompatible gene delivery tools for plants.⁵⁰ We evaluated protoplast viability of PAH-TMGMV coated with GT₁₅-Cy3 (0.1-0.5 mg/mL) or pDNA (0.04 mg/mL) using fluorescein diacetate (FDA),⁵¹ a lipophilic fluorescent dye that is permeable to membranes of living cells. Following endogenous esterase-mediated enzymatic activity, nonfluorescent FDA is transformed into fluorescein, a green fluorescence compound. Broken cells lack esterases rendering them devoid of fluorescein signal. The FDA-treated protoplast cells were analyzed by confocal microscopy imaging and viable cell percentages were calculated based on the fluorescein presence. Both PAH-TMGMV-GT₁₅-Cy3 or PAH-TMGMV-pDNA treated and control (untreated) protoplasts showed bright green fluorescence characteristic of fluorescein and normal morphology (FIG. 5A, FIG. 15). Approximately 71%±3.5 of cells remained viable after exposure to PAH-TMGMV-GT₁₅-Cy3 (0.1 mg/mL), while increased concentrations resulted in a gradual reduction in fluorescein signal and increased number of broken cells. A dramatic reduction in fluorescein signal in protoplasts was observed after exposure to PAH-TMGMV-GT₁₅-Cy3 (0.5 mg/mL), in which almost no viable cells were observed (FIG. 5C). For protoplasts exposed to PAH-TMGMV:pDNA mass ratio (1:3), approximately 74%±3.0 of cells remained viable, which is not significantly different from the viability of untreated protoplasts (FIG. 5D, and FIG. 15). In contrast, when PAH-TMGMV was loaded with pDNA at the mass ratios of 1:6 and 1:12, significant decreases were observed in cell viability, approximately 65%±5.5 (P<0.039) at the 1:6 ratio and 43%±8.5 (P<0.0003) at the 1:12 ratio cells were viable when compared to the protoplasts-only cells (FIG. 5D, and FIG. 15). The PAH-TMGMV-pDNA concentration in this protoplast viability assay was kept similar to that used in transformation analysis (0.04 mg/mL). These findings suggest that increased loading of pDNA onto PAH-TMGMV can affect plant cell viability. Overall, our results indicate that DNA coated PAH-TMGMV are highly biocompatible with plant cells causing only a reduction of 14%±9.0 (P<0.05) in cell viability for PAH-TMGMV-GT₁₅-Cy3 and 18%±3.5 (P<0.05) for PAH-TMGMV-pDNA (1:6) compared to untreated protoplasts.

### CONCLUSIONS

We engineered plant virus-based nanocarriers (PAH-TMGMV) for facile DNA delivery into the plant cell nucleus without mechanical or biological aid with the highest loading and biocompatibility of DNA nanocarriers for plant cells reported to date. We demonstrated this approach using PAH-TMGMV that spontaneously delivered a transgene (GFP) encoded in an expression vector (pDNA) into the plant protoplast nucleus. While in this work, we used native TMGMV, some applications may require inactivated viruses to prevent plant infections⁵² Future research will assess the applicability of this approach for stable plant tissue transformation or genome editing. TMGMV may prove to be a promising tool for the delivery of genes, small-interfering RNA (siRNA) and clustered regularly interspaced short palindromic repeats (CRISPR) in plants for gene editing applications. Targeted delivery approaches could be implemented for TMGMV-mediated gene delivery into plastid genomes including coating with targeting peptides⁵³ for gene delivery to plant chloroplasts,^{20,53} and mitochondria.²¹ This nanotechnology approach utilizing PAH-TMGMV for DNA delivery paves the way for developing plant virus-based nanocarriers with tunable and well controlled properties,^{37,38,54} cost-effectiveness, scalability,^{55,56} degradability,⁵⁴, and high biocompatibility.^{54,57} that enable a more sustainable agriculture and advance plant biotechnology.

### Materials and Methods

### Plant Growth

Arabidopsis thaliana Columbia ecotype (Col-0; ABRC seed stock no: CS60000) plant seedlings were grown in (2.5 in.×2.5 in.×3 in.) pots filled with soil containing 1% Marathon and 1% Osmocote in growth chambers (Conviron, Adaptis 1000). Growth chamber conditions were 200 μmol m⁻² s⁻¹ PAR with continuous 22° C. and 16/8 h day/night cycle.

### Isolation of Arabidopsis Plant Protoplasts

Protoplasts were isolated and purified following a previously described protocol¹ with modifications. Briefly, approximately 5-10 g of leaf tissues from 2- to 3-week-old seedlings were soaked in ˜20 mL of an enzyme solution [(0.25% (w/v) macerozyme R-10, 1.0% (w/v) cellulase R-10, 400 mM mannitol, 8 mM CaCl₂, and 5 mM MES-KOH, pH 5.6)] and incubated for ˜8-12 h with gentle agitation at ˜22-23° C. in the dark. After the enzymatic digestion of the plant cell wall, the protoplast suspension was filtered through a 100-μm mesh filter to remove debris, washed with 30 mL of W5 solution [2 mM MES, 154 mM NaCl, 125 mM CaCl₂, and 5 mM KCl (pH 5.6)], and centrifuged for 6 minutes at 100 g. Finally, the 1 mL of protoplast suspension was loaded onto ˜5 mL of an ice cold 21% sucrose solution in a 15-mL Falcon tube and centrifuged at 730 r.p.m (98×g) for 10 minutes in a swinging-bucket rotor centrifuge (Allegra X-3R, Beckman Coulter) to collect the purified protoplast fractions. A 10 μL of protoplasts were diluted with a W5 buffer and counted using a hemocytometer counting chamber with a microscope, resulting in a cell density of ˜1.8×10⁵ cells mL⁻¹.

### Synthesis of TMGMV Nanocarriers

TMGMV particles were obtained from BioProdex, Inc. (Gainesville, FL, USA). The resuspended particles were dialyzed for 72 hours in 10 mM potassium phosphate buffer, pH 7.0, and subsequently ultracentrifuged at 42,000×g, 2.30 hours with 40% sucrose (Beckman Coulter Optima L-90k Ultracentrifuge with 50.2 Ti rotor, Brea, CA, USA). The resulting pellets were resuspended overnight in Milli-Q water, lyophilized, and stored as TMGMV powder at −20° C. until use. The concentration was determined using a Nanodrop 2000 (Thermo Scientific, Waltham, MA, USA) and the TMGMV-specific extinction coefficient ε_{260 nm}=3.0 mL mg-1 cm⁻¹.

PAH was coated onto TMGMV using carbodiimide chemistry. The reaction conditions were optimized following previous studies with modifications.² Briefly, 2 mg of lyophilized TMGMV particles were dissolved in 1 mL of MES buffer (10 mM MES with 0.01% Triton-X-100, pH 6.0). Then, 10 mM EDC and 17 mM sulfo-NHS were added dropwise separately and thoroughly mixed by pipetting. The mixture was incubated for 30 minutes on a rotator (R2024 Roto-mini plus, Benchmark Scientific) at ambient temperature. Subsequently, 7.4 mM of PAH (Sigma-Aldrich, Cat #283215) dissolved in 10 mM MES (pH 6.0) and added to the reaction mixture, followed by overnight incubation (16 h) at ambient temperature with gentle agitation on the rotator. The TMGMV particles coated with PAH were purified by dialysis in 10 mM MES (pH 6.0) using a 100 kDa MWCO (molecular weight cutoff) dialysis membrane for 48 h by changing buffer 4 times. This process effectively removed unreacted chemicals, PAH, and broken particles. Finally, aggregated particles were removed by centrifugation at 12,000×g, 5 minutes, and the supernatant was collected. Beer-Lambert's Law was used to determine the concentration of PAH-TMGMV using a UV/visible spectrophotometer and the molar extinction coefficient, ε_{260 nm}=3.0 mL mg⁻¹ cm⁻¹.³

### Characterization of Nanomaterials

The TMGMV, PAH, PAH-TMGMV, GT₁₅-Cy3, PAH-TMGMV-GT₁₅-Cy3, and PAH-TMGMV-pDNA were characterized using absorbance UV-vis spectroscopy (UV-2600 Shimadzu), and hydrodynamic size and (potential were measured using a Zetasizer Ultra (Malvern Panalytical). The (potential and hydrodynamic diameter of the nanomaterials were measured in a 10 mM MES buffer (pH 6.0) and used 0.1 mM NaCl to enhance conductivity followed by the Hückel approximation model. The fluorescence emission from PAH-TMGMV-GT₁₅-Cy3 was collected using a fluorescence spectrometer (Horiba PTI QM-400). The PAH coating to TMGMV was analyzed using FTIR (Thermo Nicolet 6700 FTIR).

### DNA Loading Assay by Agarose Gel Electrophoresis

The single-stranded DNA oligonucleotides GT₁₅ covalently linked with 5′ Cy3 fluorophore (Cy3-GTGTGTGTGTGTGTGTGTGTGTGTGTGTGT), were purchased from Integrated DNA Technologies, Inc. (Iowa, USA). For loading GT₁₅-Cy3 onto TMGMV or PAH-TMHMV, approximately 0.5 mg of TMGMV or PAH-TMHMV was mixed with 0.1 mg of GT₁₅-Cy3 in 1 mL of 10 mM MES buffer at pH 6.0. The mixture was incubated at ambient temperature with gentle agitation for 30 minutes to facilitate electrostatic binding. After incubation, the sample was purified using a centrifugal filter unit with a 100 kDa MWCO (Cat #UFC510096). Samples were washed four times using 500 μL of 10 mM MES buffer at pH 6.0, followed by centrifugation at 12,000×g for 10 minutes each time to remove unreacted GT₁₅-Cy3. Both the eluent and washout samples were measured using a UV-vis spectrophotometer to further confirm the absence of GT₁₅-Cy3 in the sample. Beer-Lambert's Law was used to determine the concentration of PAH-TMGMV conjugated with GT₁₅-Cy3 using a UV/visible spectrophotometer (UV-2600 Shimadzu) and the molar extinction coefficient, ε_{260 nm}=3.0 mL mg⁻¹ cm⁻¹.³

The p35S-eGFP plasmid⁴ (RRID: Addgene_80127) was obtained from Addgene. It contains open reading frames (ORF) encoding the GFP gene, nuclear-specific CaMV35S constitutive promoter and the nopaline synthase terminator (Nos-T). The p35S-eGFP plasmid was purified from transformed E. coli DH5α cell culture using ZymoPURE II Plasmid Midiprep Kit (Zymo Research, CA, USA) following the manufacturer's protocols. The p35S-eGFP concentration was measured by Nanodrop (2000c) spectrophotometers (Thermo Scientific, Waltham, MA, USA). For loading p35S-eGFP onto PAH-TMGMV, 0.1 μg of PAH-TMGMV was mixed with various concentrations of p35S-eGFP (0.1, 0.3, 0.6, 1.2, 2.0, and 3.0 μg) in total a 20 μL buffer of 10 mM MES (pH 6.0). This mixture was allowed to incubate at room temperature for 30 minutes to facilitate electrostatic binding. To assess the binding of p35S-eGFP to PAH-TMGMV, 1% TBE agarose gel electrophoresis was running in 1×TBE buffer at 120 V for 60 minutes, and DNA bands were visualized using SYBR-Safe (Cat #S33102) gel stain and imaged by an Azure Biosystems 200 gel imager.

### Transmission Electron Microscopy

A volume of 10 μL (0.5 mg/mL) of TMGMV or PAH-TMGMV were applied to Formvar carbon film-coated copper TEM grids (FCF400-CU, Electron Microscopy Sciences) and allowed to dry at room temperature. The grids were rinsed three times, each time for 30 seconds, with 15 μL of deionized water to remove excess buffer and analyte. Subsequently, 15 μL of 2% (w/v) uranyl acetate was added to the grid for an additional 90 s, after which excess staining solution was removed using filter paper. Samples were examined at 200 kV using a FEI Tecnai F30 transmission electron microscope.

### Confocal Fluorescence Microscopy

Arabidopsis protoplasts, exposed to nanomaterials or untreated, were imaged using laser scanning confocal microscopy (TCS SP5, Leica Microsystems, Germany) employing a ×40 wet objective lens (Leica Microsystems, Germany). A 20 μL of protoplast cell suspension, both treated with nanomaterials and untreated, were gently placed and mounted onto glass slides within pre-made wells containing an observation gel (Carolina). For confocal analysis of Cy3 and PAH-TMGMV-GT₁₅-Cy3, a 543 nm laser was used as excitation source (at 40% power), and the photomultiplier tube (PMT) was set to detect emissions within the 550-590 nm range. For cell viability assay, fluorescein dye (FDA) was excited with a 488 nm laser (at 40% power), and emissions were detected within the 500-550 nm range using PMT. For imaging GFP expression by confocal microscopy, we utilized a 488 nm laser excitation and detected fluorescence emissions within the 500-530 nm range. For cell staining, FM-4-64 was excited with a 543 nm laser and the emission spectrum of FM-4-64 was in the range of 580-650 nm. Hoechst-33342 and chloroplast autofluorescence was examined with 405 nm laser excitation and emissions were detected at the following wavelength ranges: 420-480 nm (Hoechst-33342), and 720-780 nm (chloroplast autofluorescence). All images were acquired with a focal plane pinhole size set to 1 airy unit.

### DNA Delivery in Isolated Protoplasts

A volume of 100 μL of the protoplast in the W5 solution, containing 1.8×10⁵ cells mL-1, was treated with 0.1 mg/mL of PAH-TMGMV-GT₁₅-Cy3 at ambient temperature for 2 hours. The supernatant, which contained excess free PAH-TMGMV-GT₁₅-Cy3, was carefully removed without breaking the protoplast pellet and immediately resuspended in 100 μL of MMG solution (0.4 M mannitol, 15 mM MgCl₂, and 4 mM MES, pH 5.7). For nucleus and cell membrane staining, immediately before imaging the protoplasts, Hoechst-33342 (10 μg/mL) and FM4-64 (1 μg/mL) were added to the cell suspension for 10-15 minutes at ambient temperature. Subsequently, the cells were washed and imaged by confocal microscopy (TCS SP5, Leica Microsystems, Germany). The images were overlaid using ImageJ software for internalization analysis.

For protoplasts transformation, 100 μL of isolated protoplasts diluted with MMG buffer (1.5×10⁵ cells mL-1) was added to 25 g of p35S-eGFP loaded with 4.1 g of PAH-TMGMV (0.041 mg/mL) or only 25 g of p35S-eGFP and 4.1 g of PAH-TMGMV as a control. The mixture was gently mixed by tapping the tube and then incubated at ambient temperature for 4 h. Subsequently, the protoplasts mixture was centrifuged at 100 g for 2 minutes to pellet the protoplasts, and the supernatant was removed. Finally, the protoplasts were resuspended in 1 mL of W5 solution and incubated for 24 hours in the dark. Confocal microscopy (TCS SP5, Leica Microsystems, Germany) employing a ×40 wet objective lens (Leica Microsystems, Germany) was used to image the protoplasts.

### Denaturing Polyacrylamide Gel Electrophoresis (SDS-NuPAGE)

A 20 g of TMGMV, PAH-TMGMV, and PAH-TMGMV-GT₁₅-Cy3 were denatured using 4×LDS loading dye (NP0008, Life Technologies) for 100° C. for 5 minutes. The TMGMV proteins and the Precision Plus Protein Kaleidoscope Standard (161-0375, Bio-Rad) were separated using 12% NuPAGE in-house casting gel in 1×MOPS buffer (NP0001-02, Life Technologies) for 40 minutes at 200 V and 120 mA. Subsequently, the gels were stained with Coomassie Blue (0.25% w/v) and imaged using an Azure Biosystems 300 imaging system under white light.

### Protein Extraction and Western Blotting

To extract total soluble proteins, transformed or non-transformed protoplasts were harvested in microcentrifuge tubes, centrifuged at 100 g for 3 minutes to pellet the protoplasts, and the supernatant was removed. A 300 μL of protein extraction buffer (50 mM Tris-HCl, pH 7.5, 150 mM NaCl, 1 mM DTT, 0.1% [v/v] Triton X-100, and a protease inhibitor cocktail) was added to the cell pellets and mixed by pipetting. The protoplast mixture was sonicated for cell lysis using a sonicator at output amplitude 1 for 10 seconds. Subsequently, it was centrifuged at 3,000×g at 4° C. for 10 minutes, and the supernatant was collected as the total soluble fraction and protein concentrations were determined by Pierce BCA protein assay kits (Thermo Scientific).

For western blotting, 30 g of total soluble proteins obtained from transformed or non-transformed protoplasts were denatured using a 6×SDS sample buffer and heated at 100° C. for 5 minutes. The proteins were then separated by 12% sodium dodecyl sulfate-polyacrylamide gel electrophoresis (SDS-PAGE) under reducing conditions. Proteins were transferred to PVDF membranes using the Bio-Rad Trans-Blot Turbo Transfer System 690BR, following the manufacturer's protocol. The membrane was incubated in a blocking buffer consisting of 5% non-fat dried milk in TBST buffer (20 mM Tris-HCl, pH 7.5, 500 mM NaCl, 0.05% Tween-20) overnight at 4° C. Following the overnight incubation, the membrane was incubated with an anti-GFP antibody (Qiagen, Valencia, CA) at a dilution of 1:1000 in TBST with 2.5% non-fat dry milk for 2 h at ambient temperature and subsequently washed three times with TBST. Then, the membrane was incubated with a 1:10,000 dilution of anti-rabbit IgG conjugated with HRP (Calbiochem, San Diego, CA, USA) as a secondary antibody. Following three washing, the western blot bands were developed using azure chemiluminescent substrate (Cat #AC2101) and images were captured with an Azure Biosystems 300 imaging system.

### Plant Cell Viability Assay

The biocompatible concentration of PAH-TMGMV-GT₁₅-Cy3 and PAH-TMGMV-pDNA, and viability of isolated protoplasts was determined by the fluorescein diacetate (FDA) staining.⁵ A volume of 100 μL of isolated protoplast in W5 solution (pH 5.6), containing 1.4×10⁵ cells mL⁻¹, was treated with of PAH-TMGMV-GT₁₅-Cy3 in concentrations ranging from 0.1 mg/mL to 0.5 mg/mL or 0.04 mg/mL of PAH-TMGMV loaded with various concentration of pDNA (PAH-TMGMV:pDNA mass ratios 1:3, 1:6, 1:12 w w) at ambient temperature for 4 hours. Subsequently, the protoplasts mixture was centrifuged at 100 g for 2 minutes to pellet the protoplasts, and the supernatant was carefully discarded without breaking the protoplast pellet. The protoplasts were immediately resuspended in 100 μL of W5 solution. FDA was dissolved in acetone (5 mg/mL), and 10 μL of this solution was added to 1 mL of W5 solution to create a working solution. For staining, 100 μL of the protoplast suspensions treated with PAH-TMGMV-GT₁₅-Cy3 and PAH-TMGMV-pDNA or untreated were added to an equal volume of FDA working solution, gently mixed, and incubated at ambient temperature for 10 minutes. Subsequently, FDA-treated protoplasts were imaged using confocal microscopy (TCS SP5, Leica Microsystems, Germany) employing a ×40 wet objective lenses (Leica Microsystems, Germany). Protoplast cell viability was calculated as the percentage of round, bright, and evident green fluorescing protoplasts out of the total number of protoplasts.

### REFERENCES IN EXAMPLE 1

(1) Alexandratos, N.; Bruinsma, J. World Agriculture towards 2030/2050: The 2012 Revision. 2012.
(2) Sedeek, K. E. M.; Mahas, A.; Mahfouz, M. Plant Genome Engineering for Targeted Improvement of Crop Traits. Front. Plant Sci. 2019, 10, 114.
(3) Daniell, H.; Datta, R.; Varma, S.; Gray, S.; Lee, S. B. Containment of Herbicide Resistance through Genetic Engineering of the Chloroplast Genome. Nat. Biotechnol. 1998, 16 (4), 345-348.
(4) Liu, Y.; Wu, H.; Chen, H.; Liu, Y.; He, J.; Kang, H.; Sun, Z.; Pan, G.; Wang, Q.; Hu, J.; Zhou, F.; Zhou, K.; Zheng, X.; Ren, Y.; Chen, L.; Wang, Y.; Zhao, Z.; Lin, Q.; Wu, F.; Zhang, X.; Guo, X.; Cheng, X.; Jiang, L.; Wu, C.; Wang, H.; Wan, J. A Gene Cluster Encoding Lectin Receptor Kinases Confers Broad-Spectrum and Durable Insect Resistance in Rice. Nat. Biotechnol. 2015, 33 (3), 301-305.
(5) van Esse, H. P.; Reuber, T. L.; van der Does, D. Genetic Modification to Improve Disease Resistance in Crops. New Phytol. 2020, 225 (1), 70-86.
(6) Liu, X.; Ao, K.; Yao, J.; Zhang, Y.; Li, X. Engineering Plant Disease Resistance against Biotrophic Pathogens. Curr. Opin. Plant Biol. 2021, 60, 101987.
(7) Zhang, H.; Zhu, J.; Gong, Z.; Zhu, J.-K. Abiotic Stress Responses in Plants. Nat. Rev. Genet. 2022, 23 (2), 104-119.
(8) Saijo, Y.; Loo, E. P.-I. Plant Immunity in Signal Integration between Biotic and Abiotic Stress Responses. New Phytol. 2020, 225 (1), 87-104.
(9) Srivastav, V. K.; Egbuna, C.; Tiwari, M. Chapter 1—Plant Secondary Metabolites as Lead Compounds for the Production of Potent Drugs. In Phytochemicals as Lead Compounds for New Drug Discovery; Egbuna, C., Kumar, S., Ifemeje, J. C., Ezzat, S. M., Kaliyaperumal, S., Eds.; Elsevier, 2020; pp 3-14.
(10) Wolfson, W. Grow Your Own: Protalix BioTherapeutics Produces Drugs in Carrot Cells. Chem. Biol. 2013, 20 (8), 969-970.
(11) Daniell, H.; Nair, S. K.; Esmaeili, N.; Wakade, G.; Shahid, N.; Ganesan, P. K.; Islam, M. R.; Shepley-McTaggart, A.; Feng, S.; Gary, E. N.; Ali, A. R.; Nuth, M.; Cruz, S. N.; Graham-Wooten, J.; Streatfield, S. J.; Montoya-Lopez, R.; Kaznica, P.; Mawson, M.; Green, B. J.; Ricciardi, R.; Milone, M.; Harty, R. N.; Wang, P.; Weiner, D. B.; Margulies, K. B.; Collman, R. G. Debulking SARS-CoV-2 in Saliva Using Angiotensin Converting Enzyme 2 in Chewing Gum to Decrease Oral Virus Transmission and Infection. Mol. Ther. 2022, 30 (5), 1966-1978.
(12) Paolino, K. M.; Regules, J. A.; Moon, J. E.; Ruck, R. C.; Bennett, J. W.; Remich, S. A.; Mills, K. T.; Lin, L.; Washington, C. N.; Fornillos, G. A.; Lindsey, C. Y.; O'Brien, K. A.; Shi, M.; Mark Jones, R.; Green, B. J.; Tottey, S.; Chichester, J. A.; Streatfield, S. J.; Yusibov, V. Safety and Immunogenicity of a Plant-Derived Recombinant Protective Antigen (rPA)-Based Vaccine against Bacillus Anthracis: A Phase 1 Dose-Escalation Study in Healthy Adults. Vaccine 2022, 40 (12), 1864-1871.
(13) Charland, N.; Gobeil, P.; Pillet, S.; Boulay, I.; Seguin, A.; Makarkov, A.; Heizer, G.; Bhutada, K.; Mahmood, A.; Trepanier, S.; Hager, K.; Jiang-Wright, J.; Atkins, J.; Saxena, P.; Cheng, M. P.; Vinh, D. C.; Boutet, P.; Roman, F.; Van Der Most, R.; Ceregido, M. A.; Dionne, M.; Tellier, G.; Gauthier, J.-S.; Essink, B.; Libman, M.; Haffizulla, J.; Frechette, A.; D′Aoust, M.-A.; Landry, N.; Ward, B. J. Safety and Immunogenicity of an AS03-Adjuvanted Plant-Based SARS-CoV-2 Vaccine in Adults with and without Comorbidities. NPJ Vaccines 2022, 7 (1), 142.
(14) Herrera-Estrella, L.; Depicker, A.; Van Montagu, M.; Schell, J. Expression of Chimaeric Genes Transferred into Plant Cells Using a Ti-Plasmid-Derived Vector. 1983. Biotechnology 1992, 24, 377-381.
(15) Klein, T. M.; Harper, E. C.; Svab, Z.; Sanford, J. C.; Fromm, M. E.; Maliga, P. Stable Genetic Transformation of Intact Nicotiana Cells by the Particle Bombardment Process. Proc. Natl. Acad. Sci. U.S.A. 1988, 85 (22), 8502-8505.
(16) Shrawat, A. K.; Lorz, H. Agrobacterium-Mediated Transformation of Cereals: A Promising Approach Crossing Barriers. Plant Biotechnol. J 2006, 4 (6), 575-603.
(17) Liu, J.; Nannas, N. J.; Fu, F.-F.; Shi, J.; Aspinwall, B.; Parrott, W. A.; Dawe, R. K. Genome-Scale Sequence Disruption Following Biolistic Transformation in Rice and Maize. Plant Cell 2019, 31 (2), 368-383.
(18) Baltes, N. J.; Gil-Humanes, J.; Voytas, D. F. Genome Engineering and Agriculture: Opportunities and Challenges. Prog. Mol. Biol. Transl. Sci. 2017, 149, 1-26.
(19) Demirer, G. S.; Zhang, H.; Matos, J. L.; Goh, N. S.; Cunningham, F. J.; Sung, Y.; Chang, R.; Aditham, A. J.; Chio, L.; Cho, M.-J.; Staskawicz, B.; Landry, M. P. High Aspect Ratio Nanomaterials Enable Delivery of Functional Genetic Material without DNA Integration in Mature Plants. Nat. Nanotechnol. 2019, 14 (5), 456-464.
(20) Santana, I.; Jeon, S.-J.; Kim, H-I.; Islam, M. R.; Castillo, C.; Garcia, G. F. H.; Newkirk, G. M.; Giraldo, J. P. Targeted Carbon Nanostructures for Chemical and Gene Delivery to Plant Chloroplasts. ACS Nano 2022, 16 (8), 12156-12173.
(21) Law, S. S. Y.; Liou, G.; Nagai, Y.; Giménez-Dejoz, J.; Tateishi, A.; Tsuchiya, K.; Kodama, Y.; Fujigaya, T.; Numata, K. Polymer-Coated Carbon Nanotube Hybrids with Functional Peptides for Gene Delivery into Plant Mitochondria. Nat. Commun. 2022, 13 (1), 2417.
(22) Mitter, N.; Worrall, E. A.; Robinson, K. E.; Li, P.; Jain, R. G.; Taochy, C.; Fletcher, S. J.; Carroll, B. J.; Lu, G. Q. M.; Xu, Z. P. Clay Nanosheets for Topical Delivery of RNAi for Sustained Protection against Plant Viruses. Nat Plants 2017, 3, 16207.
(23) Li, S.; Li, J.; Du, M.; Deng, G.; Song, Z.; Han, H. Efficient Gene Silencing in Intact Plant Cells Using siRNA Delivered By Functional Graphene Oxide Nanoparticles. Angew. Chem. Int. Ed Engl. 2022, 61 (40), e202210014.
(24) Wang, J. W.; Cunningham, F. J.; Goh, N. S.; Boozarpour, N. N.; Pham, M.; Landry, M. P. Nanoparticles for Protein Delivery in Planta. Curr. Opin. Plant Biol. 2021, 60, 102052.
(25) Chariou, P. L.; Steinmetz, N. F. Delivery of Pesticides to Plant Parasitic Nematodes Using Tobacco Mild Green Mosaic Virus as a Nanocarrier. ACS Nano 2017, 11 (5), 4719-4730.
(26) Caparco, A. A.; González-Gamboa, I.; Hays, S. S.; Pokorski, J. K.; Steinmetz, N. F. Delivery of Nematicides Using TMGMV-Derived Spherical Nanoparticles. Nano Lett. 2023, 23 (12), 5785-5793.
(27) Solanki, P.; Bhargava, A.; Chhipa, H.; Jain, N.; Panwar, J. Nano-Fertilizers and Their Smart Delivery System. In Nanotechnologies in Food and Agriculture; Rai, M., Ribeiro, C., Mattoso, L., Duran, N., Eds.; Springer International Publishing: Cham, 2015; pp 81-101.
(28) Borgatta, J.; Shen, Y.; Tamez, C.; Green, C.; Hedlund Orbeck, J. K.; Cahill, M. S.; Protter, C.; Deng, C.; Wang, Y.; Elmer, W.; White, J. C.; Hamers, R. J. Influence of CuO Nanoparticle Aspect Ratio and Surface Charge on Disease Suppression in Tomato (Solanum Lycopersicum). J. Agric. Food Chem. 2023. https://doi.org/10.1021/acs.jafc.2c09153.
(29) Kwak, S.-Y.; Lew, T. T. S.; Sweeney, C. J.; Koman, V. B.; Wong, M. H.; Bohmert-Tatarev, K.; Snell, K. D.; Seo, J. S.; Chua, N.-H.; Strano, M. S. Chloroplast-Selective Gene Delivery and Expression in Planta Using Chitosan-Complexed Single-Walled Carbon Nanotube Carriers. Nat. Nanotechnol. 2019, 14 (5), 447-455.
(30) Chang, F.-P.; Kuang, L.-Y.; Huang, C.-A.; Jane, W.-N.; Hung, Y.; Hsing, Y.-I. C.; Mou, C.-Y. A Simple Plant Gene Delivery System Using Mesoporous Silica Nanoparticles as Carriers. J. Mater. Chem. B Mater. Biol. Med. 2013, 1 (39), 5279-5287.
(31) Torney, F.; Trewyn, B. G.; Lin, V. S.-Y.; Wang, K. Mesoporous Silica Nanoparticles Deliver DNA and Chemicals into Plants. Nat. Nanotechnol. 2007, 2 (5), 295-300.
(32) Li, J.; Li, S.; Du, M.; Song, Z.; Han, H. Nuclear Delivery of Exogenous Gene in Mature Plants Using Nuclear Location Signal and Cell-Penetrating Peptide Nanocomplex. ACS Appl. Nano Mater. 2023, 6 (1), 160-170.
(33) Jones, I.; Roy, P. Small Is Beautiful: Virus-like Particles as Vaccines. Biochem. 2021, 43 (4), 18-21.
(34) Pattanayek, R.; Stubbs, G. Structure of the U2 Strain of Tobacco Mosaic Virus Refined at 3.5 A Resolution Using X-Ray Fiber Diffraction. J. Mol. Biol. 1992, 228 (2), 516-528.
(35) Charudattan, R. Use of Plant Viruses as Bioherbicides: The First Virus-Based Bioherbicide and Future Opportunities. Pest Manag. Sci. 2023. https://doi.org/10.1002/ps.7760.
(36) Chariou, P. L.; Wang, L.; Desai, C.; Park, J.; Robbins, L. K.; von Recum, H. A.; Ghiladi, R. A.; Steinmetz, N. F. Let There Be Light: Targeted Photodynamic Therapy Using High Aspect Ratio Plant Viral Nanoparticles. Macromol. Biosci. 2019, 19 (5), e1800407.
(37) Fan, X. Z.; Pomerantseva, E.; Gnerlich, M.; Brown, A.; Gerasopoulos, K.; McCarthy, M.; Culver, J.; Ghodssi, R. Tobacco Mosaic Virus: A Biological Building Block for Micro/nano/bio Systems. J. Vac. Sci. Technol. A 2013, 31 (5), 050815.
(38) González-Gamboa, I.; Caparco, A. A.; McCaskill, J. M.; Steinmetz, N. F. Bioconjugation Strategies for Tobacco Mild Green Mosaic Virus. Chembiochem 2022, 23 (18), e202200323.
(39) Abrahamian, P.; Hammond, R. W.; Hammond, J. Plant Virus-Derived Vectors: Applications in Agricultural and Medical Biotechnology. Annu Rev Virol 2020, 7 (1), 513-535.
(40) Scholthof, K.-B. G.; Mirkov, T. E.; Scholthof, H. B. Plant Virus Gene Vectors: Biotechnology Applications in Agriculture and Medicine. Genet. Eng. 2002, 24, 67-85.
(41) Rössner, C.; Lotz, D.; Becker, A. VIGS Goes Viral: How VIGS Transforms Our Understanding of Plant Science. Annu. Rev. Plant Biol. 2022, 73, 703-728.
(42) Jian, W.; Xu, S.; Wang, J.; Feng, S. Layer-by-Layer Assembly of Poly(allylamine Hydrochloride)/polyurethane and Its Loading and Release Behavior for Methylene Orange. J. Appl. Polym. Sci. 2013, 129 (4), 2070-2075.
(43) Kreke, M. R.; Badami, A. S.; Brady, J. B.; Akers, R. M.; Goldstein, A. S. Modulation of Protein Adsorption and Cell Adhesion by Poly(allylamine Hydrochloride) Heparin Films. Biomaterials 2005, 26 (16), 2975-2981.
(44) Giraldo, J. P.; Landry, M. P.; Kwak, S.-Y.; Jain, R. M.; Wong, M. H.; Iverson, N. M.; Ben-Naim, M.; Strano, M. S. A Ratiometric Sensor Using Single Chirality Near-Infrared Fluorescent Carbon Nanotubes: Application to In Vivo Monitoring. Small 2015, 11 (32), 3973-3984.
(45) Lew, T. T. S.; Wong, M. H.; Kwak, S.-Y.; Sinclair, R.; Koman, V. B.; Strano, M. S. Rational Design Principles for the Transport and Subcellular Distribution of Nanomaterials into Plant Protoplasts. Small 2018, 14 (44), e1802086.
(46) He, Z.; Liu, K.; Manaloto, E.; Casey, A.; Cribaro, G. P.; Byrne, H. J.; Tian, F.; Barcia, C.; Conway, G. E.; Cullen, P. J.; Curtin, J. F. Cold Atmospheric Plasma Induces ATP-Dependent Endocytosis of Nanoparticles and Synergistic U373MG Cancer Cell Death. Sci. Rep. 2018, 8 (1), 5298.
(47) Giraldo, J. P.; Landry, M. P.; Faltermeier, S. M.; McNicholas, T. P.; Iverson, N. M.; Boghossian, A. A.; Reuel, N. F.; Hilmer, A. J.; Sen, F.; Brew, J. A.; Strano, M. S. Plant Nanobionics Approach to Augment Photosynthesis and Biochemical Sensing. Nat. Mater. 2014, 13 (4), 400-408.
(48) Wong, M. H.; Misra, R. P.; Giraldo, J. P.; Kwak, S.-Y.; Son, Y.; Landry, M. P.; Swan, J. W.; Blankschtein, D.; Strano, M. S. Lipid Exchange Envelope Penetration (LEEP) of Nanoparticles for Plant Engineering: A Universal Localization Mechanism. Nano Lett. 2016, 16 (2), 1161-1172.
(49) Lee, D. W.; Hwang, I. Transient Expression and Analysis of Chloroplast Proteins in Arabidopsis Protoplasts. In Chloroplast Research in Arabidopsis: Methods and Protocols, Volume I, Jarvis, R. P., Ed.; Humana Press: Totowa, NJ, 2011; pp 59-71.
(50) Wang, X.; Xie, H.; Wang, P.; Yin, H. Nanoparticles in Plants: Uptake, Transport and Physiological Activity in Leaf and Root. Materials 2023, 16 (8), 3097. https://doi.org/10.3390/ma16083097.
(51) Wang, H.; Zhu, X.; Li, H.; Cui, J.; Liu, C.; Chen, X.; Zhang, W. Induction of Caspase-3-like Activity in Rice Following Release of Cytochrome-F from the Chloroplast and Subsequent Interaction with the Ubiquitin-Proteasome System. Sci. Rep. 2014, 4, 5989.
(52) Chariou, P. L.; Ma, Y.; Hensley, M.; Rosskopf, E. N.; Hong, J. C.; Charudattan, R.; Steinmetz, N. F. Inactivated Plant Viruses as an Agrochemical Delivery Platform. ACS Agric. Sci. Technol. 2021, 1 (3), 124-130.
(53) Santana, I.; Wu, H.; Hu, P.; Giraldo, J. P. Targeted Delivery of Nanomaterials with Chemical Cargoes in Plants Enabled by a Biorecognition Motif. Nat. Commun. 2020, 11 (1), 2045.
(54) Koudelka, K. J.; Pitek, A. S.; Manchester, M.; Steinmetz, N. F. Virus-Based Nanoparticles as Versatile Nanomachines. Annu Rev Virol 2015, 2 (1), 379-401.
(55) Chung, Y. H.; Church, D.; Koellhoffer, E. C.; Osota, E.; Shukla, S.; Rybicki, E. P.; Pokorski, J. K.; Steinmetz, N. F. Integrating Plant Molecular Farming and Materials Research for next-Generation Vaccines. Nat Rev Mater 2022, 7 (5), 372-388.
(56) Rybicki, E. P. Plant Molecular Farming of Virus-like Nanoparticles as Vaccines and Reagents. Wiley Interdiscip. Rev. Nanomed. Nanobiotechnol. 2020, 12 (2), e1587.
(57) Kim, K. R.; Lee, A. S.; Kim, S. M.; Heo, H. R.; Kim, C. S. Virus-like Nanoparticles as a Theranostic Platform for Cancer. Front Bioeng Biotechnol 2022, 10, 1106767.

### ADDITIONAL REFERENCES IN THE MATERIALS AND METHODS SECTION

(1) Lee, D. W.; Hwang, I. Transient Expression and Analysis of Chloroplast Proteins in Arabidopsis Protoplasts. Methods Mol. Biol. 2011, 774, 59-71.
(2) Kwak, S.-Y.; Lew, T. T. S.; Sweeney, C. J.; Koman, V. B.; Wong, M. H.; Bohmert-Tatarev, K.; Snell, K. D.; Seo, J. S.; Chua, N.-H.; Strano, M. S. Chloroplast-Selective Gene Delivery and Expression in Planta Using Chitosan-Complexed Single-Walled Carbon Nanotube Carriers. Nat. Nanotechnol. 2019, 14 (5), 447-455.
(3) Chariou, P. L.; Steinmetz, N. F. Delivery of Pesticides to Plant Parasitic Nematodes Using Tobacco Mild Green Mosaic Virus as a Nanocarrier. ACS Nano 2017, 11 (5), 4719-4730.
(4) Fujii, Y.; Kodama, Y. In Planta Comparative Analysis of Improved Green Fluorescent Proteins with Reference to Fluorescence Intensity and Bimolecular Fluorescence Complementation Ability. Plant Biotechnol. 2015, 32 (1), 81-87.
(5) Wang, H.; Zhu, X.; Li, H.; Cui, J.; Liu, C.; Chen, X.; Zhang, W. Induction of Caspase-3-like Activity in Rice Following Release of Cytochrome-F from the Chloroplast and Subsequent Interaction with the Ubiquitin-Proteasome System. Sci. Rep. 2014, 4, 5989.

### Example 2. Inactivated iTMGMV-PAH-Mediated pDNA Delivery and Gene Expression In Vivo

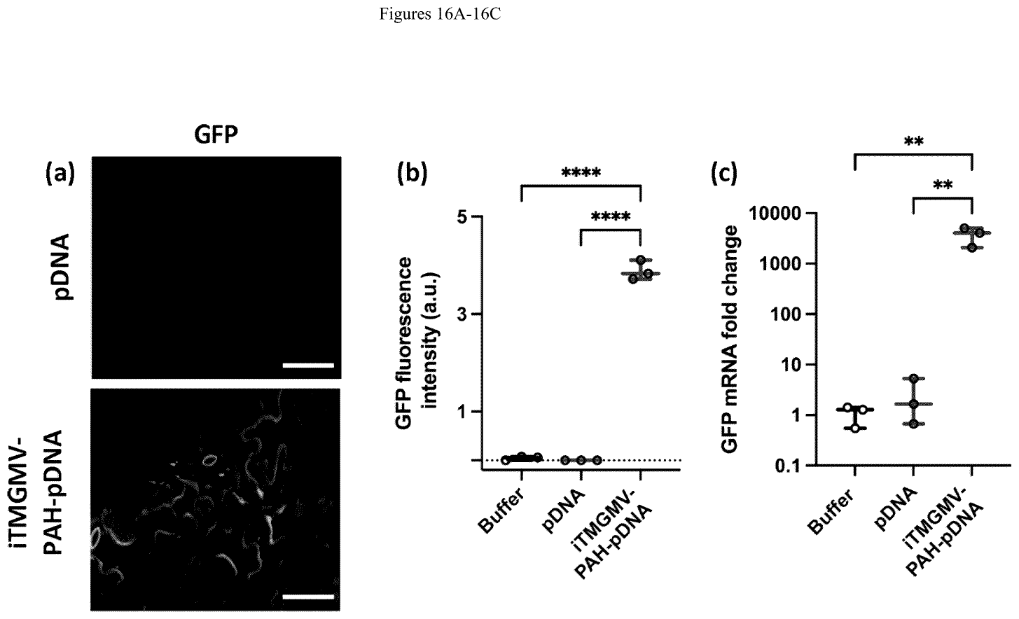

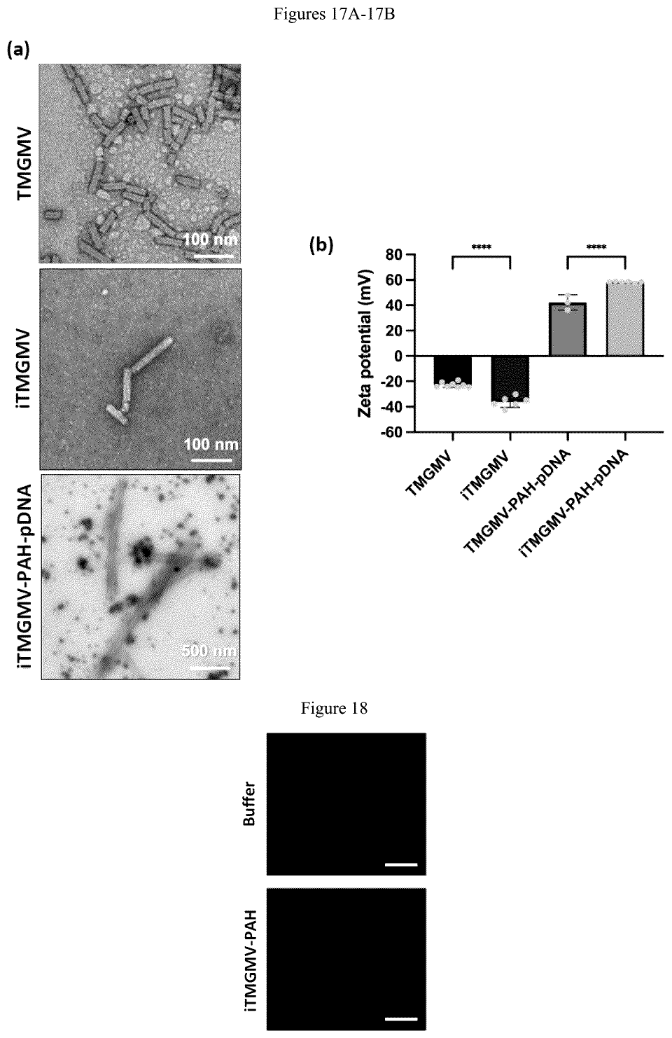

For GFP expression analysis in vivo, we inactivated TMGMV to prevent plant infection using UV light exposure as reported previously.¹ The TEM size of inactivated iTMGMV (110.73±30.22 nm) is similar to those of active TMGMV (129.9±57.7 nm) (P>0.05) (FIG. 17). In contrast, the zeta potential of iTMGMV is more negative (−36.29±4.23 mV) compared to that of active TMGMV (−22.4±2.3 mV) (10 mM MES Buffer, pH 6.0) (P<0.0001). This resulted in iTMGMV-PAH-pDNA having a higher zeta potential (58.53±0.50 mV) than TMGMV-PAH-pDNA (42.16±5.1 mV; P<0001). We abaxially infiltrated the inactivated iTMGMV-PAH coated in pDNA into 3-week-old Arabidopsis leaves at the previously established 1:6 mass loading ratio. Confocal microscopy analysis indicated that 0.1 mg/mL of iTMGMV-PAH bound to 0.6 mg/mL of pDNA can enable GFP expression into leaf epidermal cells (FIG. 16A). Buffer or iTMGMV-PAH infiltrated leaves did not exhibit GFP fluorescence (FIG. 18). Leaves infiltrated with iTMGMVPAH-pDNA had a high GFP fluorescence intensity (FIG. 16B). RT-qPCR analysis quantifying GFP mRNA fold change expression supported GFP expression mediated by 0.1 mg/mL iTMGMV-PAH:0.6 mg/mL pDNA (FIG. 16C). Together, these analyses show that (i)TMGMV-PAHs have the highest pDNA mass loading ratio for nanocarriers reported to date, preserve and protect the pDNA integrity from degradation, and facilitate spontaneous pDNA translocation across the plant plasma membrane and cell wall, enabling transgene expression in the nucleus in vitro and in vivo.

Maintaining cell viability after exposure to nanocarriers with DNA is crucial for enabling biocompatible gene delivery tools for plants.² We evaluated protoplast viability of TMGMV-PAH coated with GT15-Cy3 (0.1-0.5 mg/mL) or pDNA (0.04 mg/mL) using fluorescein diacetate (FDA),³ a lipophilic fluorescent dye that is permeable to membranes of living cells. Following endogenous esterase-mediated enzymatic activity, nonfluorescent FDA is transformed to fluorescein, a green fluorescence compound. Broken cells lack esterases, rendering them devoid of fluorescein signal. The FDA-treated protoplast cells were analyzed by confocal microscopy imaging, and viable cell percentages were calculated based on the fluorescein presence. Both TMGMV-PAH-GT15-Cy3 or TMGMV-PAH-pDNA treated and control (untreated) protoplasts showed bright green fluorescence characteristic of fluorescein and normal morphology (FIGS. 5A-5B). Approximately 71%±3.5 of cells remained viable after exposure to TMGMV-PAH-GT15-Cy3 (0.1 mg/mL), while increased concentrations resulted in a gradual reduction in fluorescein signal and increased number of broken cells (FIG. 5C). A dramatic reduction in the fluorescein signal in protoplasts was observed after exposure to TMGMV-PAH-GT15-Cy3 (0.5 mg/mL), in which almost no viable cells were observed (FIG. 5C). For protoplasts exposed to the TMGMV-PAH:pDNA mass ratio (1:3), approximately 74%±3.0 of cells remained viable, which is not significantly different from the viability of untreated protoplasts (FIG. 5D). In contrast, when TMGMV-PAH was loaded with pDNA at the mass ratios of 1:6 and 1:12, significant decreases were observed in cell viability, approximately 65%±5.5 (P<0.039) at the 1:6 ratio and 43%±8.5 (P<0.0003) at the 1:12 ratio cells were viable when compared to the protoplasts-only cells (FIG. 5D). The TMGMVPAH-pDNA concentration in this protoplast viability assay was kept similar to that used in the transformation analysis (0.04 mg/mL). These findings suggest that an increased loading of pDNA onto TMGMV-PAH can affect plant cell viability.

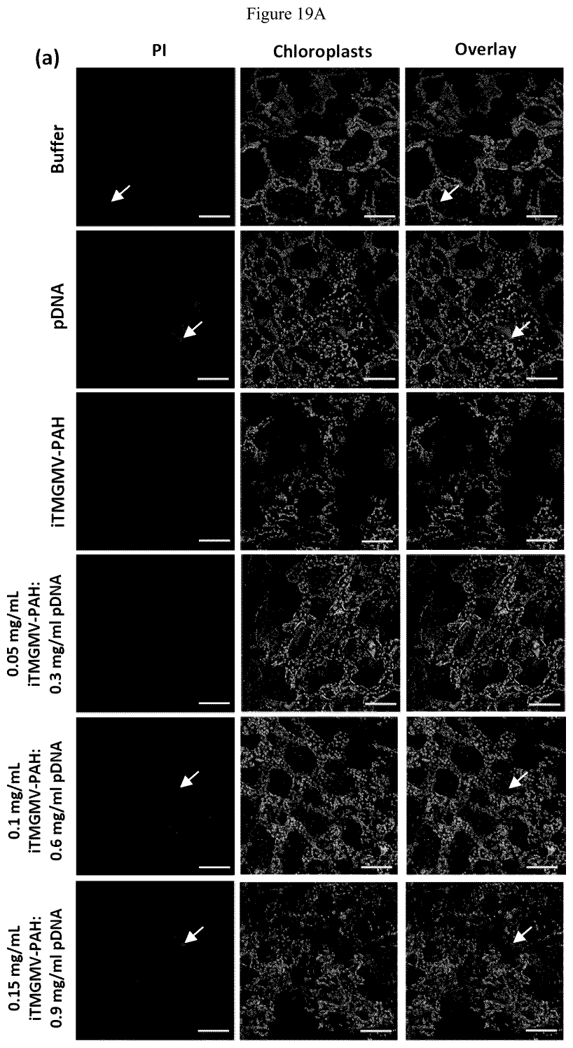

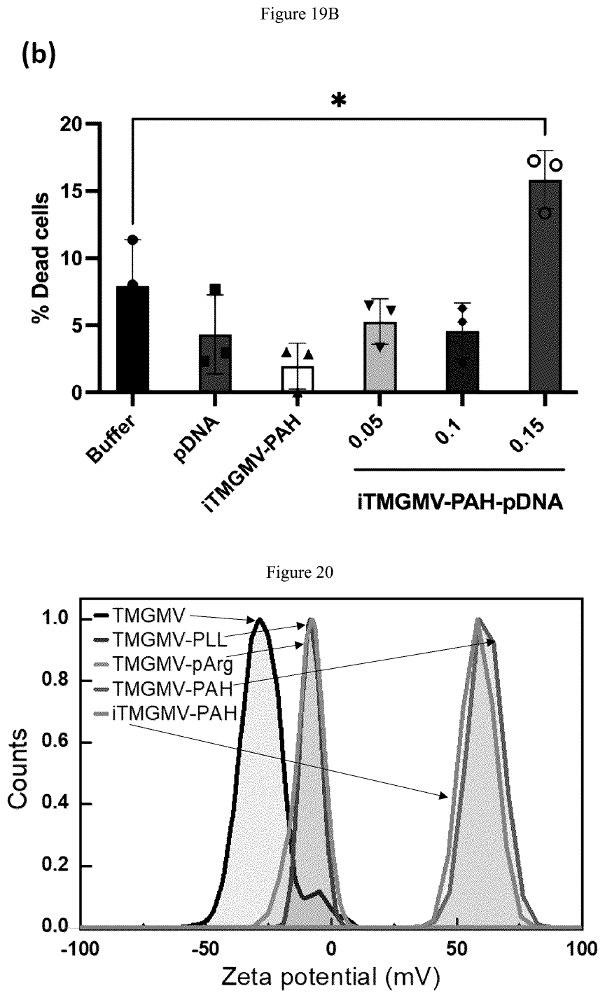

Biocompatibility of iTMGMV-PAH-pDNA in Arabidopsis leaves was determined using propidium iodide, a fluorescent dye that stains the nucleus of dead cells (FIG. 19). Confocal microscopy images of leaf cells infiltrated with our chosen concentration for GFP expression analysis of 0.1 mg/mL iTMGMV-PAH: 0.6 mg/mL pDNA showed a similar percentage of dead cells (4.5±1.7%) to leaves treated with buffer control (7.9±3.4%; P>0.5; FIGS. 19A-19B). Higher concentrations of 0.15 mg/mL iTMGMV-PAH: 0.9 mg/mL pDNA significantly increased the percentage of dead cells (15.8±2.2%; P<0.01). Overall, our results indicate that DNA coated TMGMV-PAH are highly biocompatible with plant cells both in vitro in plant protoplasts and in vivo in leaf cells.

### Methods:

Synthesis of TMGMV and iTMGMV Nanocarriers

The iTMGMV nanocarriers were synthesized using the same method described in Example 1, except TMGMV-PAH were inactivated (iTMGMV-PAH) by UV crosslinking as described in a previous study (Chariou, et al., ACS Agric. Sci. Technol. 2021, 1 (3), 124-130).

### DNA Delivery in Plant Cells

For DNA delivery in vivo, 3 week old Arabidopsis thaliana leaves were gently abaxially infiltrated with 40 μl of the iTMGMV-PAH-pDNA or control solutions (10 mM MES pH 6.0 buffer, pDNA, iTMGMV-PAH) using a 1 mL needleless syringe. Plants were returned to the growth chamber for 2 days before being imaged with a Leica SP5 confocal microscope.

### Confocal Fluorescence Microscopy

For imaging GFP expression by confocal microscopy in vitro and in vivo, we utilized a 488 nm laser excitation and detected fluorescence emissions within the 500-530 nm range. For in vivo GFP imaging analysis, leaf punches were taken from each treatment 2 days post-infiltration and mounted onto a glass slide as described above. Confocal images were taken 2 days post-infiltration and analyzed to quantify GFP expression. Three biological replicates were performed for each treatment and analyzed with ImageJ analysis to quantify GFP fluorescence intensity values. Fluorescence intensity was calculated using the corrected total cell fluorescence (CTCF) equation. CTCF=Integrated Density−(Area of selected cell×Mean Fluorescence of Background readings). The CTCF was calculated for each biological replicate and correlated to the GFP expression in the sample.

### RT-qPCR Analysis

Leaves were collected 2 days post infiltration with iTMGMV-PAH-pDNA or control solutions (10 mM MES pH 6.0 buffer, pDNA). Total RNA was extracted using the Zymo Research Quick RNA Plant Miniprep Kit (Cat #R2024) with DNase treatment (Zymo Research DNase I Set (Cat #E1010). One-step RT-qPCR was performed to quantify GFP expression in Arabidopsis leaves using the NEB Luna Universal One-Step RT-qPCR Kit (Cat #E3005) with 10 ng total RNA and 0.25 nM primer templates. Thermocycler conditions were as follows: 55° C. for 10 minutes, 95° C. for 5 minutes, and 35 cycles of 95° C. for 10 seconds and 50° C. for 30 seconds with a plate read. The melt curve was run from 65-95° C. in 0.5° C. increments. qPCR data was analyzed by the ddCt method to obtain normalized GFP mRNA fold change with respect to the ACTIN-2 housekeeping gene. Three technical replicates were performed and three biological replicates were collected for each treatment.

### Leaf Cell Viability Assay

Cell viability staining of infiltrated Arabidopsis thaliana leaves was performed using 0.1 mM propidium iodide (PI), a fluorescent dye that stains the nucleus of dead cells. Briefly, leaves were infiltrated with buffer (10 mM MES pH 6.0), 0.6 mg/mL pDNA, 0.1 mg/mL iTMGMV-PAH, 0.15 mg/mL iTMGMV-PAH: 0.9 mg/mL pDNA, 0.1 mg/mL iTMGMV-PAH: 0.6 mg/mL pDNA, and 0.05 mg/mL iTMGMV-PAH: 0.3 mg/mL pDNA. At 2 days post infiltration, leaf discs were stained with PI for 30 min on a rotating mixer. Confocal images were detected at ranges of 590-640 nm for PI and 720-780 nm for chloroplasts under laser excitation of 488 nm.

### REFERENCES IN EXAMPLE 2

1. Chariou, P. L.; Ma, Y.; Hensley, M.; Rosskopf, E. N.; Hong, J. C.; Charudattan, R.; Steinmetz, N. F. Inactivated Plant Viruses as an Agrochemical Delivery Platform. ACS Agric. Sci. Technol. 2021, 1 (3), 124-130.
2. Wang, X.; Xie, H.; Wang, P.; Yin, H. Nanoparticles in Plants: Uptake, Transport and Physiological Activity in Leaf and Root. Materials 2023, 16 (8), 3097.
3. Wang, H.; Zhu, X.; Li, H.; Cui, J.; Liu, C.; Chen, X.; Zhang, W. Induction of Caspase-3-like Activity in Rice Following Release of Cytochrome-F from the Chloroplast and Subsequent Interaction with the Ubiquitin-Proteasome System. Sci. Rep. 2014, 4, 5989.

The entire content of MR Islam et al., Nano Lett. 2024, 24, 26, 7833-7842 are incorporated by reference herein.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A nanoparticle, comprising a cationic polymer conjugated to the nanoparticle, wherein the nanoparticle is a live virus, or an inactivated virus.

2. The nanoparticle of claim 1, wherein the nanoparticle is selected from the group consisting of tobacco mosaic virus, tobacco mild green mosaic virus (TMGMV), cowpea mosaic virus, and potato virus X.

3. The nanoparticle of claim 1, wherein the cationic polymer is poly-(allylamine) or salt thereof.

4. The nanoparticle of claim 3, wherein the poly-(allylamine) or salt thereof has a molecular weight of at least about 10 kDa.

5. The nanoparticle of claim 1, wherein the nanoparticle has an aspect ratio that is greater than 3.

6. The nanoparticle of claim 1, wherein the nanoparticle has a length of about 60-600 nm.

7. The nanoparticle of claim 1, wherein the nanoparticle has a width of about 15-300 nm.

8. The nanoparticle of claim 1, wherein the nanoparticle has a Zeta potential of about 50-65 mV.

9. The nanoparticle of claim 1, wherein a nucleic acid is adsorbed on the nanoparticle outer surface.

10. The nanoparticle of claim 9, wherein the nucleic acid is DNA.

11. The nanoparticle of claim 9, wherein the nucleic acid is RNA.

12. The nanoparticle of claim 9, wherein the nanoparticle has a Zeta potential of about 10-60 mV.

13. The nanoparticle of claim 9, wherein the mass ratio of nanoparticle to nucleic acid is about 10:1 to 1:12.

14. The nanoparticle of claim 13, wherein the mass ratio of nanoparticle to nucleic acid is about 1:3 to 1:6.

15. The nanoparticle of claim 1, wherein the nanoparticle is TMGMV.

16. The nanoparticle of claim 1, wherein the nanoparticle is a live virus.

17. The nanoparticle of claim 1, wherein the nanoparticle is an inactivated virus.

18. The nanoparticle of claim 15, which comprises a Tobacco mild green mosaic virus (TMGMV) coat protein (CP) that is conjugated with poly-(allylamine) or salt thereof.

19. The nanoparticle of claim 18, wherein the poly-(allylamine) conjugated coat protein has a molecular weight of at least about 30 kDa.

20. A method for delivering nucleic acid to plant cell(s), comprising contacting the plant cell(s) with a nanoparticle according to claim 9.

21. The method of claim 20, wherein the plant cell(s) is contacted in vitro.

22. The method of claim 20, wherein the plant cell(s) is contacted in vivo.

23. The method of claim 22, wherein the nanoparticle is an inactivated virus.

24. The method of claim 23, wherein the viability of plant cells after contacting is at least about 80% or higher.
